SEQUENCE LISTING (39 sequences)

SEQ ID NO: 1 - protein, 219 aa, Escherichia coli
  misc_feature (1)..(219): >yghB
MAVIQDIIAALWQHDFAALADPHIVSVVYFVMFATLFLENGLLPASFLPGDSLLILAGALIAQGVMDFLPTIAILTAAASLGCWLSYIQGRWLGNTKTVKGWLAQLPAKYHQRATCMFDRHGLLALLAGRFLAFVRTLLPTMAGISGLPNRRFQFFNWLSGLLWVSVVTSFGYALSMIPFVKRHEDQVMTFLMILPIALLTAGLLGTLFVVIKKKYCNA

SEQ ID NO: 2 - protein, 289 aa, Escherichia coli
  misc_feature (1)..(289): >robA
MDQAGIIRDLLIWLEGHLDQPLSLDNVAAKAGYSKWHLQRMFKDVTGHAIGAYIRARRLSKSAVALRLTARPILDIALQYRFDSQQTFTRAFKKQFAQTPALYRRSPEWSAFGIRPPLRLGEFTMPEHKFVTLEDTPLIGVTQSYSCSLEQISDFRHEMRYQFWHDFLGNAPTIPPVLYGLNETRPSQDKDDEQEVFYTTALAQDQADGYVLTGHPVMLQGGEYVMFTYEGLGTGVQEFILTVYGTCMPMLNLTRRKGQDIERYYPAEDAKAGDRPINLRCELLIPIRR

SEQ ID NO: 3 - protein, 221 aa, Escherichia coli
  misc_feature (1)..(221): >marC
MLDLFKAIGLGLVVLLPLANPLTTVALFLGLAGNMNSAERNRQSLMASVYVFAIMMVAYYAGQLVMDTFGISIPGLRIAGGLIVAFIGFRMLFPQQKAIDSPEAKSKSEELEDEPSANIAFVPLAMPSTAGPGTIAMIISSASTVRQSSTFADWVLMVAPPLIFFLVAVILWGSLRSSGAIMRLVGKGGIEAISRLMGFLLVCMGVQFIINGILEIIKTYH

SEQ ID NO: 4 - protein, 413 aa, Escherichia coli
  misc_feature (1)..(413): >fabF
MSKRRVVVTGLGMLSPVGNTVESTWKALLAGQSGISLIDHFDTSAYATKFAGLVKDFNCEDIISRKEQRKMDAFIQYGIVAGVQAMQDSGLEITEENATRIGAAIGSGIGGLGLIEENHTSLMNGGPRKISPFFVPSTIVNMVAGHLTIMYGLRGPSISIATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDAGIEASQIGYVNAHGTSTPAGDKAEAQAVKTIFGEAASRVLVSSTKSMTGHLLGAAGAVESIYSILALRDQAVPPTINLDNPDEGCDLDFVPHEARQVSGMEYTLCNSFGFGGTNGSLIFKKI

SEQ ID NO: 5 - protein, 356 aa, Escherichia coli
  misc_feature (1)..(356): >plsX
MTRLTLALDVMGGDFGPSVTVPAALQALNSNSQLTLLLVGNSDAITPLLAKADFEQRSRLQIIPAQSVIASDARPSQAIRASRGSSMRVALELVKEGRAQACVSAGNTGALMGLAKLLLKPLEGIERPALVTVLPHQQKGKTVVLDLGANVDCDSTMLVQFAIMGSVLAEEVVEIPNPRVALLNIGEEEVKGLDSIRDASAVLKTIPSINYIGYLEANELLTGKTDVLVCDGFTGNVTLKTMEGVVRMFLSLLKSQGEGKKRSWWLLLLKRWLQKSLTRRFSHLNPDQYNGACLLGLRGTVIKSHGAANQRAFAVAIEQAVQAVQRQVPQRIAARLESVYPAGFELLDGGKSGTLR

SEQ ID NO: 6 - protein, 161 aa, Escherichia coli
  misc_feature (1)..(161): >rraA
MKYDTSELCDIYQEDVNVVEPLFSNFGGRASFGGQIITVKCFEDNGLLYDLLEQNGRGRVLVVDGGGSVRRALVDAELARLAVQNEWEGLVIYGAVRQVDDLEELDIGIQAMAAIPVGAAGEGIGESDVRVNFGGVTFFSGDHLYADNTGIILSEDPLDIE

SEQ ID NO: 7 - protein, 483 aa, Escherichia coli
  misc_feature (1)..(483): >trkH
MHFRAITRIVGLLVILFSGTMIIPGLVALIYRDGAGRAFTQTFFVALAIGSMLWWPNRKEKGELKSREGFLIVVLFWTVLGSVGALPFIFSESPNLTITDAFFESFSGLTTTGATTLVGLDSLPHAILFYRQMLQWFGGMGIIVLAVAILPILGVGGMQLYRAEMPGPLKDNKMRPRIAETAKTLWLIYVLLTVACALALWFAGMDAFDAIGHSFATIAIGGFSTHDASIGYFDSPTINTIIAIFLLISGCNYGLHFSLLSGRSLKVYWRDPEFRMFIGVQFTLVVICTLVLWFHNVYSSALMTINQAFFQVVSMATTAGFTTDSIARWPLFLPVLLLCSAFIGGCAGSTGGGLKVIRILLLFKQGNRELKRLVHPNAVYSIKLGNRALPERILEAVWGFFSAYALVFIVSMLAIIATGVDDFSAFASVVATLNNLGPGLGVVADNFTSMNPVAKWILIANMLFGRLEVFTLLVLFTPTFWRE

SEQ ID NO: 8 - protein, 162 aa, Escherichia coli
  misc_feature (1)..(162): >iscR
MRLTSKGRYAVTAMLDVALNSEAGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTRTQDAIDVKLRA

SEQ ID NO: 9 - protein, 91 aa, Escherichia coli [sequence omitted]

SEQ ID NO: 10 - DNA, 46 bp, Artificial Sequence
  Synthetic Polynucleotide >yghB expr fwd
ggataacaatttcacacatactagtcgctgttccacaggaaagtcc

SEQ ID NO: 11 - DNA, 50 bp, Artificial Sequence
  Synthetic Polynucleotide >yghB expr rev
ctttcgttttatttgatgcctggtacctaggccgggaacggggaaaatcg

SEQ ID NO: 12 - DNA, 50 bp, Artificial Sequence
  Synthetic Polynucleotide >rob del fwd
aattacctgatgtcaggtgctcgttgttgaaaggatgaggatattttatg

SEQ ID NO: 13 - DNA, 50 bp, Artificial Sequence
  Synthetic Polynucleotide >rob del rev
gacgcccctgcattagatgagctgcagcgttaacgacggatcggaatcag

SEQ ID NO: 14 - DNA, 50 bp, Artificial Sequence
  Synthetic Polynucleotide >marC fwd
cttatacttttcgctgataacccagatacacaggataacaaccaccaatg

SEQ ID NO: 15 - DNA, 50 bp, Artificial Sequence
  Synthetic Polynucleotide >marC rev
aatagttgaaaggcccattcgggccttttttaatggtacgttttaatgat

SEQ ID NO: 16 - DNA, 50 bp, Artificial Sequence
  Synthetic Polynucleotide >yghB del fwd
gtacaataggcagataaaggcttaaacgctgttccacaggaaagtccatg

SEQ ID NO: 17 - DNA, 50 bp, Artificial Sequence
  Synthetic Polynucleotide >yghB del rev
cggtacagcaaccgggaacgggaaaatcgtcaggcgttacagtatttttt

SEQ ID NO: 18 - DNA, 50 bp, Artificial Sequence
  Synthetic Polynucleotide >fabF del fwd
tctttttgtcccactagaatcattttttccctccctggaggacaaacgtg

SEQ ID NO: 19 - DNA, 50 bp, Artificial Sequence
  Synthetic Polynucleotide >fabF del rev
gaccttttataagggtggaaaatgacaacttagatctttttaaagatcaa

SEQ ID NO: 20 - DNA, 50 bp, Artificial Sequence
  Synthetic Polynucleotide >plsX del fwd
tttccccaggcaactggggaaagaccaaaccgggcggcgacgataccttg

SEQ ID NO: 21 - DNA, 50 bp, Artificial Sequence
  Synthetic Polynucleotide >plsX del rev
caaactgcgagttcgctggcagcgtcctgctaccgcagagttccgctttt

SEQ ID NO: 22 - DNA, 45 bp, Artificial Sequence
  Synthetic Polynucleotide >rob H fwd
ggataacaatttcacacatactagtcctgatgtcaggtgctcgtt

SEQ ID NO: 23 - DNA, 54 bp, Artificial Sequence
  Synthetic Polynucleotide >rob H rev
ctttcgttttatttgatgcctggtacctaggtcacgaataccaaaggcgctcca

SEQ ID NO: 24 - DNA, 49 bp, Artificial Sequence
  Synthetic Polynucleotide >marC35 fwd
ggataacaatttcacacatactagttacacaggataacaaccaccaatg

SEQ ID NO: 25 - DNA, 51 bp, Artificial Sequence
  Synthetic Polynucleotide >marC35 rev
ctttcgttttatttgatgcctggtacctaggtcagttgcctgccaggccaa

SEQ ID NO: 26 - DNA, 50 bp, Artificial Sequence
  Synthetic Polynucleotide >P yghB delta Homology fwd
tttattgtgaaaagtcttaaattgtcgtcccggacgattcaggagtacaa

SEQ ID NO: 27 - DNA, 20 bp, Artificial Sequence
  Synthetic Polynucleotide >P yghB del Hom Amp - rev
cagcgtggcaaacatgacaa

SEQ ID NO: 28 - DNA, 20 bp, Artificial Sequence
  Synthetic Polynucleotide >P yghB del Hom Amp - fwd
tctgattgccgatctggacg

SEQ ID NO: 29 - DNA, 20 bp, Artificial Sequence
  Synthetic Polynucleotide >P yghB del check - rev
cagcagcgtacggacaaatg

SEQ ID NO: 30 - DNA, 21 bp, Artificial Sequence
  Synthetic Polynucleotide >P yghB del check - fwd
tcttaaattgttgcgtcccgg

SEQ ID NO: 31 - DNA, 36 bp, Artificial Sequence
  Synthetic Polynucleotide >P yghB CRISPR 20 nt rev
ctatttctagctctaaaacggatcaaggcgtcccgg

SEQ ID NO: 32 - DNA, 34 bp, Artificial Sequence
  Synthetic Polynucleotide >P yghB CRISPR 20 nt fwd
taatacgactcactatagcgtccgggacgccttg

SEQ ID NO: 33 - DNA, 20 bp, Artificial Sequence
  Synthetic Polynucleotide >Datsenko k1
cagtcatagccgaatagcct

SEQ ID NO: 34 - DNA, 20 bp, Artificial Sequence
  Synthetic Polynucleotide >Datsenko k2
cggtgccctgaatgaactgc

SEQ ID NO: 35 - DNA, 50 bp, Artificial Sequence
  Synthetic Polynucleotide >rraA Keio 1
atagcgcgatatactgaaaattctcgcagcaactgaatgttaagcctatg

SEQ ID NO: 36 - DNA, 50 bp, Artificial Sequence
  Synthetic Polynucleotide >rraA Keio 2
aaaaaaggcaccttgcggtgcctttcttatcattcaatatccagcggatc

SEQ ID NO: 37 - DNA, 22 bp, Artificial Sequence
  Synthetic Polynucleotide >YghB Check Short Rev
gcgtttaagcctttatctgcct

SEQ ID NO: 38 - DNA, 24 bp, Artificial Sequence
  Synthetic Polynucleotide >YghB Check Short Fwd
acttttggacaattttgcagacat

SEQ ID NO: 39 - DNA, 50 bp, Artificial Sequence
  Synthetic Polynucleotide >Pyghb 50 bp Homology
ttattgtgaaaagtcttaaattgttgcgtcccggacgattcaggagtaca

(19) United States
(12) Patent Application Publication
(10) Pub. No.: US 2023/0041211 A1
(43) Pub. Date: Feb. 9, 2023

(54) DECREASING TOXICITY OF TERPENES AND INCREASING THE PRODUCTION POTENTIAL IN MICRO-ORGANISMS

(71) Applicant: BASF SE, Ludwigshafen an Rhein (DE)

(72) Inventors: Michael BREUER, Ludwigshafen am Rhein (DE); Julia Ena HARTIG, Oslo (NO); Robert THUMMER, Ludwigshafen am Rhein (DE); Heike BRUESER, Ludwigshafen am Rhein (DE); Stephanie RENZ, Ludwigshafen am Rhein (DE); Michael Guenter BRAUN, Ludwigshafen am Rhein (DE); Oliver OSWALD, Ludwigshafen am Rhein (DE); Roland MINGES, Minden (DE); Jens O. KROEMER, Leipzing (DE); Heiko BABEL, Leipzing (DE)

(21) Appl. No.: 17/786,127

(22) PCT Filed: Dec. 15, 2020
(86) PCT No.: PCT/EP2020/086120

§ 371 Date: Jun. 16, 2022

(30) Foreign Application Priority Data
Dec. 20, 2019 (EP) 19218714.4

(51) Int. Cl. C12N 15/01; C12N 1/20; C12N 15/74; C12N 15/70; C12P 5/00

(52) U.S. Cl. CPC C12N 15/01; C12N 1/20; C12N 15/74; C12N 15/70; C12P 5/007

(57) ABSTRACT

The invention pertains to novel methods to increase the tolerance of microbial host cells to toxic substance, for example terpenes and alcohols and other membrane disrupting substances, as well as modified organism with such an increased tolerance a compared to the unmodified organism.

### BACKGROUND OF THE INVENTION

Isoprenol belongs to the class of naturally occurring terpenoid compounds (Withers and Keasling, 2006). 3-Methyl-3-buten-1-ol is the basis for the chemical production of Citral, Menthol and other flavor compounds also belonging to the terpenoid class. Citral consecutively is used for the synthesis of Vitamin A and E and several Carotenoids. Isoprenol has also been discussed as a lead nutraceutical for longevity (Pandey et al., 2019). Recently companies such as Amyris and Isobionics have introduced terpenoid products such as artemisinic acid, valencene and nootkatone that are synthesized in biotechnological fermentation processes. Those companies are currently developing biological production platforms to further expand their product portfolio in the fragrance and flavor business (Janssen, 2015) and thus challenge chemical synthesis.

Biotechnological production of terpenoid compounds in microorganisms relies on the natural precursor Isopentenyl Diphosphate (IPP) from which by simple dephosphorylation Isoprenol can be obtained. Bioengineering so far focused on increasing the intracellular concentration of the Isoprenol precursor IPP. In the model organism E. coli this has been achieved by introducing an additional metabolic pathway that produces IPP, the DXP pathway, resulting in product titers of 61 mg/L (Liu et al., 2014). If mixtures of Prenol and Isoprenol are considered as product, titers up to 1 g/L are currently possible (Kang et al., 2017). So far the toxic intermediate IPP has been identified as a major obstacle in those processes (George et al., 2018; Kang et al., 2019). Current research projects try to develop integrated processes where Isopentenol is obtained from hydrolyzed polysaccharides originating from biomass (Wang et al., 2019).

A key issue in the biotechnological production of terpenoids is their toxicity towards microorganisms (Brennan et al., 2015), it therefore is an issue that every economically viable bioprocess has to face. This issue can be overcome by using two-phase production systems as disclosed in the international patent application published as WO2015/002528 and by evolution engineering of the production strains to a higher tolerance.

Producing monoterpene esters in microorganisms has also been demonstrated. When geraniol was produced in E. coli, it was observed that the chloramphenicol acetyltransferase gene mediated formation of geranyl acetate (Liu et al. Biotechnol Biofuels (2016) 9:58). The use of more specific enzymes has been shown to bring advantages: While monoterpene alcohols such as geraniol, but also linalool which is an acyclic monoterpene found in the floral scents of many plants are very toxic to microbes, their esters are often much less toxic. Toxicity of monoterpene alcohols often leads to an arrest in growth and/or production, and thus only very low product titers have been achieved. Chacon et al. have shown that expression of RhAAT, in an E. coli which was engineered to produce geraniol, lead to formation of geranyl acetate at levels which were substantially increased relative to the levels of geraniol produced in the absence of RhAAT (Chacón, M. G., et al. Esterification of geraniol as a strategy for increasing product titre and specificity in engineered Escherichia coli. Microb Cell Fact 18, 105 (2019);

https://doi.org/10.1186/s12934-019-1130-0; WO 2019/092388). For that reason, in situ esterification of monoterpene alcohols such as geraniol has been forwarded as a means to detoxify the product and, thus, increase terpene production.

The problem to be solved was to develop host cells with and the methods for increasing tolerance to terpenoids and/or other toxic substances such as host cells better suited for Isoprenol bioproduction.

Surprisingly, some novel and unexpected modifications to host cells were found to result in a broad tolerance to terpenes and other substances.

### SUMMARY OF THE INVENTION

The invention discloses novel methods to increase the tolerance of microbial host cells to toxic substances, for example terpenes and alcohols and other membrane disrupting substances, as well as host cells with such an increased tolerance compared to the unmodified host cell.

The toxicity of the terpenes Menthol, Geraniol, Citral, Isoprenol was tested with unmodified organism of Escherichia coli, Saccharomyces cerevisae, Pseudomonas putida and Rhodobacter sphaeroides. All showed toxic effects, however especially Geraniol and Citral were strongly degraded making them less suitable for our engineering approach. To determine modifications useful in increasing the tolerance of microorganisms to these and similar toxic substances, cells of the E. coli strain MG1655 were subjected to constant growth in the presence of 60 mM Isoprenol in a way that did not kill the cells but allowed for adaptation and mutations. Then the concentration was increased from initially 60 mM (10 mM above the half maximal inhibition dose, EC50) to 80 mM Isoprenol after 80 generations to increase the selection pressure. In this concentration regime wild-type E. coli cells are not able to grow, but the adapted E. coli strains did grow, and showed even faster growth on reduced isoprenol concentrations compared to the parental E. coli. Over the course of more than 220 generations, isolates were generated for detailed analysis. Isolates from three parallel cultures (Isolate A to C) and 7 different timepoints in the evolution (T1-T7) were analysed for the modifications that are responsible for the increased tolerance. After in depth analysis the modifications considered most promising were isolated and introduced into E. coli wildtype cells and knock out cells.

Using these modifications, the growth inhibiting effects on host cells of a number of substances as disclosed herein could be decreased. The present invention therefore discloses methods of decreasing toxicity of terpenes and increasing the production potential in micro-organisms and host cells with such improved features.

### DETAILED DESCRIPTION OF THE INVENTION

The terms “essentially”, “about”, “approximately”, “substantially” and the like in connection with an attribute or a value, particularly also define exactly the attribute or exactly the value, respectively. The term “substantially” in the context of the same functional activity or substantially the same function means a difference in function preferably within a range of 20%, more preferably within a range of 10%, most preferably within a range of 5% or less compared to the reference function. In context of formulations or compositions, the term “substantially” (e.g., “composition substantially consisting of compound X”) may be used herein as containing substantially the referenced compound having a given effect within the formulation or composition, and no further compound with such effect or at most amounts of such compounds which do not exhibit a measurable or relevant effect. The term “about” in the context of a given numeric value or range relates in particular to a value or range that is within 20%, within 10%, or within 5% of the value or range given. As used herein, the term “comprising” also encompasses the term “consisting of”.

The term “isolated” means that the material is substantially free from at least one other component with which it is naturally associated within its original environment. For example, a naturally occurring polynucleotide, polypeptide, or enzyme present in a living animal is not isolated, but the same polynucleotide, polypeptide, or enzyme, separated from some or all of the coexisting materials in the natural system, is isolated. As further example, an isolated nucleic acid, e.g., a DNA or RNA molecule, is one that is not immediately contiguous with the 5′ and 3′ flanking sequences with which it normally is immediately contiguous when present in the naturally occurring genome of the organism from which it is derived. Such polynucleotides could be part of a vector, incorporated into a genome of a cell with an unrelated genetic background (or into the genome of a cell with an essentially similar genetic background, but at a site different from that at which it naturally occurs), or produced by PCR amplification or restriction enzyme digestion, or an RNA molecule produced by in vitro transcription, and/or such polynucleotides, polypeptides, or enzymes could be part of a composition, and still be isolated in that such vector or composition is not part of its natural environment.

“Purified” means that the material is in a relatively pure state, e.g., at least about 90% pure, at least about 95% pure, or at least about 98% or 99% pure. Preferably “purified” means that the material is in a 100% pure state.

A “synthetic” or “artificial” compound is produced by in vitro chemical or enzymatic synthesis. It includes, but is not limited to, variant nucleic acids made with optimal codon usage for host organisms, such as a yeast cell host or other expression hosts of choice or variant protein sequences with amino acid modifications, such as e.g. substitutions, compared to the wildtype protein sequence, e.g. to optimize properties of the polypeptide.

The term “non-naturally occurring” refers to a (poly)nucleotide, amino acid, (poly)peptide, enzyme, protein, cell, organism, or other material that is not present in its original environment or source, although it may be initially derived from its original environment or source and then reproduced by other means. Such non-naturally occurring (poly)nucleotide, amino acid, (poly)peptide, enzyme, protein, cell, organism, or other material may be structurally and/or functionally similar to or the same as its natural counterpart.

The term “native” (or “wildtype” or “endogenous”) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “heterologous” (or exogenous or foreign or recombinant) polypeptide is defined herein as:

(a) a polypeptide that is not native to the host cell. The protein sequence of such a heterologous polypeptide is a synthetic, non-naturally occurring, “man made” protein sequence;
(b) a polypeptide native to the host cell but structural modifications, e.g., deletions, substitutions, and/or insertions, are included as a result of manipulation of the DNA of the host cell by recombinant DNA techniques to alter the native polypeptide; or
(c) a polypeptide native to the host cell whose expression is quantitatively altered or whose expression is directed from a genomic location different from the native host cell as a result of manipulation of the DNA of the host cell by recombinant DNA techniques, e.g., a stronger promoter.

Descriptions b) and c), above, refer to a sequence in its natural form but not naturally expressed by the cell used for its production. The produced polypeptide is therefore more precisely defined as a “recombinantly expressed endogenous polypeptide”, which is not in contradiction to the above definition but reflects the specific situation that it's not the sequence of a protein being synthetic or manipulated but the way the polypeptide molecule is produced.

Similarly, the term “heterologous” (or exogenous or foreign or recombinant) polynucleotide refers:

(a) to a polynucleotide that is not native to the host cell;
(b) a polynucleotide native to the host cell but structural modifications, e.g., deletions, substitutions, and/or insertions, are included as a result of manipulation of the DNA of the host cell by recombinant DNA techniques to alter the native polynucleotide;
(c) a polynucleotide native to the host cell whose expression is quantitatively altered as a result of manipulation of the regulatory elements of the polynucleotide by recombinant DNA techniques, e.g., a stronger promoter; or
(d) a polynucleotide native to the host cell but integrated not within its natural genetic environment as a result of genetic manipulation by recombinant DNA techniques.

With respect to two or more polynucleotide sequences or two or more amino acid sequences, the term “heterologous” is used to characterize that the two or more polynucleotide sequences or two or more amino acid sequences do not occur naturally in the specific combination with each other.

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

For nucleotide sequences, e.g., consensus sequences, an IUPAC nucleotide nomenclature (Nomenclature Committee of the International Union of Biochemistry (NC-IUB) (1984). “Nomenclature for Incompletely Specified Bases in Nucleic Acid Sequences”) is used, with the following nucleotide and nucleotide ambiguity definitions, relevant to this invention: A, adenine; C, cytosine; G, guanine; T, thymine; K, guanine or thymine; R, adenine or guanine; W, adenine or thymine; M, adenine or cytosine; Y, cytosine or thymine; D, not a cytosine; N, any nucleotide. In addition, notation “N(3-5)” means that indicated consensus position may have 3 to 5 any (N) nucleotides. For example, a consensus sequence “AWN(4-6)” represents 3 possible variants—with 4, 5, or 6 any nucleotides at the end: AWNNNN, AWNNNNN, AWNNNNNN.

The terms “regulatory element” and “regulatory sequence” are all used interchangeably herein and are to be taken in a broad context to refer to regulatory nucleic acid sequences capable of effecting expression of the sequences to which they are associated, including but not limited thereto, the expression of a polynucleotide encoding a polypeptide. Regulatory elements or regulatory sequences may include any nucleotide sequence having a function or purpose individually and/or within a particular arrangement or grouping of other elements or sequences within the arrangement. Examples of regulatory sequences include, but are not limited to, a leader or signal sequence (such as a 5′-UTR), a start signal, a pro-peptide sequence, a promoter, an enhancer, a silencer, a polyadenylation sequence, a ribosomal binding site (RBS, shine dalgarno sequence), a stop signal, a terminator, a 3′-UTR, and combinations thereof. Regulatory elements or regulatory sequences may be native (i.e. from the same gene) or foreign (i.e. from a different gene) to each other or to a nucleotide sequence to be expressed.

The term “operably linked” means that the described components are in a relationship permitting them to function in their intended manner. For example, a regulatory sequence operably linked to a coding sequence is ligated in such a way that expression of the coding sequence is achieved under condition compatible with the regulatory sequences.

Nucleic acids and polypeptides may be modified to include tags or domains. Tags may be utilized for a variety of purposes, including for detection, purification, solubilization, or immobilization, and may include, for example, biotin, a fluorophore, an epitope, a mating factor, or a regulatory sequence. Domains may be of any size and which provides a desired function (e.g., imparts increased stability, solubility, activity, simplifies purification) and may include, for example, a binding domain, a signal sequence, a promoter sequence, a regulatory sequence, an N-terminal extension, or a C30 terminal extension. Combinations of tags and/or domains may also be utilized.

The term “fusion protein” refers to two or more polypeptides joined together by any means known in the art. These means include chemical synthesis or splicing the encoding nucleic acids by recombinant engineering.

Methods of Modification of Nucleic Acids to Introduce Changes in the Encoded Protein
Gene Editing

Gene editing or genome editing is a type of genetic engineering in which DNA is inserted, replaced, or removed from a genome and which can be obtained by using a variety of techniques such as “gene shuffling” or “directed evolution” consisting of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547), or with “T-DNA activation” tagging (Hayashi et al. Science (1992) 1350-1353), where the resulting transgenic organisms show dominant phenotypes due to modified expression of genes close to the introduced promoter, or with “TILLING” (Targeted Induced Local Lesions In Genomes) and refers to a mutagenesis technology useful to generate and/or identify nucleic acids encoding proteins with modified expression and/or activity. TILLING also allows selection of organisms carrying such mutant variants. Methods for TILLING are well known in the art (McCallum et al., (2000) Nat Biotechnol 18: 455-457; reviewed by Stemple (2004) Nat Rev Genet 5(2): 145-50). Another technique uses artificially engineered nucleases like Zinc finger nucleases, Transcription Activator-Like Effector Nucleases (TALENs), the CRISPR/Cas system, and engineered meganuclease such as re-engineered homing endonucleases (Esvelt, K M.; Wang, H H. (2013), Mol Syst Biol 9 (1): 641; Tan, W S. et al. (2012), Adv Genet 80: 37-97; Puchta, H.; Fauser, F. (2013), Int. J. Dev. Biol 57: 629-637).
Mutagenesis

DNA and the proteins that they encoded can be modified using various techniques known in molecular biology to generate variant proteins or enzymes with new or altered properties. For example, random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196.

Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793.

Alternatively, modifications, additions or deletions are introduced by error-prone PCR, shuffling, site-directed mutagenesis, assembly PCR, sexual PCR mutagenesis, in vivo mutagenesis (phage-assisted continuous evolution, in vivo continuous evolution), cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

Alternatively, “gene site saturation mutagenesis” or “GSSM” includes a method that uses degenerate oligonucleotide primers to introduce point mutations into a polynucleotide, as described in detail in U.S. Pat. Nos. 6,171,820 and 6,764,835.

Alternatively, Synthetic Ligation Reassembly (SLR) includes methods of ligating oligonucleotide building blocks together non-stochastically (as disclosed in, e.g., U.S. Pat. No. 6,537,776). Alternatively, Tailored multi-site combinatorial assembly (“TMSCA”) is a method of producing a plurality of progeny polynucleotides having different combinations of various mutations at multiple sites by using at least two mutagenic non-overlapping oligonucleotide primers in a single reaction. (as described in PCT Pub. No. WO 2009/018449).

Sequence alignments can be generated with a number of software tools, such as:
Needleman and Wunsch algorithm—Needleman, Saul B. & Wunsch, Christian D. (1970). “A general method applicable to the search for similarities in the amino acid sequence of two proteins”. Journal of Molecular Biology. 48 (3): 443-453.

This algorithm is, for example, implemented into the “NEEDLE” program, which performs a global alignment of two sequences. The NEEDLE program, is contained within, for example, the European Molecular Biology Open Software Suite (EMBOSS).
EMBOSS—a collection of various programs: The European Molecular Biology Open Software Suite (EMBOSS), Trends in Genetics 16 (6), 276 (2000).
BLOSUM (BLOcks SUbstitution Matrix)—typically generated on the basis of alignments of conserved regions, e.g. of protein domains (Henikoff S, Henikoff JG: Amino acid substitution matrices from protein blocks. Proceedings of the National Academy of Sciences of the USA. 1992 Nov. 15; 89(22):10915-9). One out of the many BLOSUMs is “BLOSUM62”, which is often the “default” setting for many programs, when aligning protein sequences.
BLAST (Basic Local Alignment Search Tool)—consists of several individual programs (BlastP, BlastN, . . . ) which are mainly used to search for similar sequence in large sequence databases. BLAST programs also create local alignments. Typically used is the “BLAST” interface provided by NCBI (National Center for Biotechnology Information), which is the improved version (“BLAST2”). The “original” BLAST: Altschul, S. F., Gish, W., Miller, W., Myers, E. W. & Lipman, D. J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “I” symbol in the alignment indicates identical residues (which means bases for DNA or amino acids for proteins). The number of identical residues is 6.

The “−” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %−identity=(identical residues/length of the alignment region which is showing the shorter sequence over its complete length)*100. Thus, sequence identity in relation to comparison of two amino acid sequences according to this embodiment is calculated by dividing the number of identical residues by the length of the alignment region which is showing the shorter sequence over its complete length. This value is multiplied with 100 to give “%−identity”. According to the example provided above, %−identity is: (6/8)*100=75%.

Variants of the santalene synthase may have an amino acid sequence which is at least n percent identical to the amino acid sequence of the respective parent polypeptide molecule with n being an integer between 50 and 100, preferably 50, 55, 60, 65, 70, 75, 80, 85, 90, 91, 92, 93, 94, 95, 96, 97, 98 or 99 compared to the full-length polypeptide sequence.

Santalene synthase variants may be defined by their sequence similarity when compared to a parent enzyme. Sequence similarity usually is provided as “% sequence similarity” or “%−similarity”. For calculating sequence similarity in a first step a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is called “conservative mutation”. Enzyme variants comprising conservative mutations appear to have a minimal effect on protein folding resulting in certain enzyme properties being substantially maintained when compared to the enzyme properties of the parent enzyme.

For determination of %−similarity according to this invention the following applies, which is also in accordance with the BLOSUM62 matrix as for example used by the “NEEDLE” program (as referenced above),

which is one of the most used amino acids similarity matrix for database searching and sequence alignments.
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

Therefore, according to the present description the following calculation of percent-similarity applies:

%−similarity=[(identical residues+similar residues)/length of the alignment region which is showing the shorter sequence over its complete length]*100. Thus, sequence similarity in relation to comparison of two amino acid sequences according to this embodiment is calculated by dividing the number of identical residues plus the number of similar residues by the length of the alignment region which is showing the shorter sequence over its complete length. This value is multiplied with 100 to give “%−similarity”.

Variant enzymes comprising conservative mutations which are at least m % similar to the respective parent sequences with m being an integer between 50 and 100, preferably 50, 55, 60, 65, 70, 75, 80, 85, 90, 91, 92, 93, 94, 95, 96, 97, 98 or 99 compared to the full-length polypeptide sequence, are expected to have essentially unchanged enzyme properties, such as enzymatic activity.

“Construct”, “genetic construct” or “expression cassette (used interchangeably) as used herein, is a DNA molecule composed of at least one sequence of interest to be expressed, operably linked to one or more regulatory sequences (at least to a promoter) as described herein. Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature message that accumulates in the cytosol. The skilled artisan is well aware of the genetic elements that must be present in the expression cassette to be successfully expressed. Preferably, at least part of the DNA or the arrangement of the genetic elements forming the expression cassette is artificial. The expression cassette may be part of a vector or may be integrated into the genome of a host cell and replicated together with the genome of its host cell. The expression cassette is capable of increasing or decreasing the expression of DNA and/or protein of interest.

The term “introduction” or “transformation” as referred to herein encompasses the transfer of an exogenous polynucleotide into a host cell, irrespective of the method used for transfer. That is, the term “transformation” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, N.Y.), but it encompasses any further kind polynucleotide transfer methods such as, but not limited to, transduction or transfection.

The term “recombinant organism” refers to a eukaryotic organism (yeast, fungus, alga, plant, animal) or to a prokaryotic microorganism (e.g., bacteria) which has been genetically altered, modified or engineered such that it exhibits an altered, modified or different genotype as compared to the wild-type organism which it was derived from. Preferably, the “recombinant organism” comprises an exogenous nucleic acid. “Recombinant organism”, “genetically modified organism” and “transgenic organism” are used herein interchangeably. The exogenous nucleic acid can be located on an extrachromosomal piece of DNA (such as plasmids) or can be integrated in the chromosomal DNA of the organism. In the case of a recombinant eukaryotic organism, it is understood as meaning that the nucleic acid(s) used are not present in, or originating from, the genome of said organism, or are present in the genome of said organism but not at their natural locus in the genome of said organism, it being possible for the nucleic acids to be expressed under the regulation of one or more endogenous and/or exogenous regulatory element.

Per definition, the term “terpenes” comprises the hydrocarbons only, being composed of carbon and hydrogen and terpene compounds. The term “terpene compound” refers to terpenes and terpenes containing additional functional groups, resulting in derivatives such as alcohols, aldehydes, ketones and acids, but also includes related compounds such as the four carbon (C4) alcohols butanol and isobutanol or the eight carbon aldehyde Vanillin. Typical terpene compounds are
those alcohols with four carbon atoms (C4), such as but not limited to butanol and isobutanol;
compounds with five carbon atoms (C5), such as but not limited to the hemiterpene isoprene and the hemiterpenoids prenol and isovaleric acid;
seven or eight carbon phenolic aldehydes like but not limited to Vanillin;
compounds with ten carbon atoms (C10) that are terpenes or derived from terpenes, or compounds derived from C10 terpenes, such as but not limited to the monoterpenes and monoterpenoids like geraniol, terpineol, limonene, myrcene, linalool or pinene;
compounds with fifteen carbon atoms (C15) that are terpenes or derived from terpenes, or compounds derived from C15 terpenes, such as but not limited to the sesquiterpenes and sesquiterpenoids like humulene, farnesenes, farnesol; and
compounds with twenty carbon atoms (C20), compounds with twenty-five carbon atoms (C25), compounds with thirty carbon atoms (C30), compounds with thirty-five carbon atoms (C35),), or compounds with forty carbon atoms (C40) that are terpenes or derived from terpenes, or compounds derived from C20, C25, C30, C35 or C40 terpenes.

In one embodiment, a terpene compound is to be understood to be a terpene; a terpene containing one or more additional functional groups, resulting in a derivative such as an alcohol, an aldehyde, an ketone or an acid; a C4 alcohol, preferably butanol or isobutanol; or Vanillin or Isovanillin. Preferably a terpene compound is a terpene with five, ten or fifteen carbon atoms or a compound derived therefrom.

With respect to monoterpene compounds, the C10 compound geranyl diphosphate (GPP) is the direct precursor in the formation of monoterpenes comprising a series of consecutive reactions including hydrolysis, cyclizations, and oxidoreductions.

There are two main types of monoterpenes: acyclic (or linear) and cyclic which can be mono- or bicyclic. Acyclic monoterpenes, such as cis-alpha-ocimene and beta-myrcene are 2,6-dimethyloctane derivatives. Typical monocyclic monoterpenes, as limonene and cymene, are, in principle, cyclohexane derivatives with an isopropyl substituent, commonly containing variable double bond moieties. alpha-Pinene and beta-pinene are, on the other hand, the common types of bicyclic monoterpenes.

“Terpene alcohols” as used herein means a terpene compound comprising an alcohol group as a functional group. Many examples are known in the art.

“Monoterpene alcohol” as used herein means a monoterpene (C10) comprising an alcohol group as a functional group. Monoterpene alcohols are well described in the art.

“Sesquiterpene alcohol” as used herein means a sesquiterpene (C15) comprising an alcohol group as a functional group. Sesquiterpene alcohols are well known in the art.

Terpene alcohols, for example monoterpene or sesquiterpene alcohols can be primary, secondary or tertiary alcohols as is known in the art.

Preferred primary alcohols are geraniol, citronellol, lavandulol and preferred secondary alcohols are borneol, isoborneol, fenchol, verbenol, carveol, menthol. Also preferred are nerolidol, santalol, cubebol, patchoulol, bisabolol, germacrene D-ol, hedycariol.

Diterpene alcohols like sclareol may also be used in the methods of the invention.

Acyclic monoterpene alcohols, or monoterpenols as sometimes referred to in literature, are 2,6-dimethyloctane derivatives containing variable double bond moieties and a hydroxyl-function. The most important substances of this class are linalool, geraniol, nerol, citronellol, myrcenol, and dihydromyrcenol. They are used in perfumery because of their pleasant olfactory properties since ancient times. The modified organisms or the methods of the invention may be used in one embodiment in production of these as well.

Per definition, an ester is a chemical compound derived from an acid (organic or inorganic) in which at least one —OH (hydroxyl) group is replaced by an —O-alkyl (alkoxy) group. A “terpene ester” hence is a terpene alcohol in which at least one —OH (hydroxyl) group is replaced by an —O-alkyl (alkoxy) group.

“Monoterpene esters” as used herein means esters from monoterpene alcohols. The term includes esters from primary monoterpene alcohols, secondary monoterpene alcohols or tertiary monoterpene alcohols, as defined herein.

“Sesquiterpene esters” as used herein means esters from sesquiterpene alcohols. The term includes esters from primary sesquiterpene alcohols, secondary sesquiterpene alcohols or tertiary sesquiterpene alcohols, as defined herein.

The invention is directed to a modified organism with improved tolerance to one or more terpene compounds, wherein the modified organism has one or more alterations compared to a wildtype modified organism selected from the following group consisting of:
i. Absence, inactivation or reduced abundance of the protein of SEQ ID NO: 2 or a homolog thereof and absence, inactivation or reduced abundance of the protein of SEQ ID NO: 3 or a homolog thereof and presence of a mutated protein of the protein of SEQ ID NO: 2 or a homolog thereof in the presence of terpene compounds, wherein the mutated protein of the protein of SEQ ID NO: 2 or a homolog thereof shares in order of preference only the first 54, 53, 52, 51, 50, 49, 48 or 47 amino acids with the protein of SEQ ID NO: 2 or homolog thereof of the non-modified organism.
ii. Absence, inactivation or reduced abundance of the protein of SEQ ID NO: 2 or a homolog thereof in the presence of terpene compounds
iii. Absence, inactivation or reduced abundance of the protein of SEQ ID NO: 3 or a homolog thereof in the presence of terpene compounds
iv. Absence of the protein of SEQ ID NO: 2 or a homolog thereof and presence of a mutated protein of the protein of SEQ ID NO: 2 or a homolog thereof in the presence of terpene compounds, wherein the mutated protein of the protein of SEQ ID NO: 2 or a homolog thereof has a mutation at the position corresponding to the position 48 of SEQ ID NO: 2;
v. Presence of a mutated protein of the protein of SEQ ID NO: 2 or a homolog thereof in the presence of terpene compounds, wherein the mutated protein of the protein of SEQ ID NO: 2 or a homolog thereof has a mutation at the position corresponding to the position 48 of SEQ ID NO: 2;
vi. Increased levels or increased activity compared to the non-modified organism of protein of SEQ ID NO: 1 or a homolog thereof in the presence of terpene compounds, preferably wherein the endogenous gene for the homolog of SEQ ID NO: 1 has been deleted and with recombinant expression of a gene encoding SEQ ID NO: 1 or a variant thereof, even more preferably wherein the recombinant expression of a gene encoding SEQ ID NO: 1 or a variant thereof is under a low to medium strength promoter or other control element;
vii. Presence of a mutated protein of the protein of SEQ ID NO: 4 or a homolog thereof in the presence of terpene compounds, wherein the mutated protein of the protein of SEQ ID NO: 4 or a homolog thereof has a mutation at the position corresponding to the position 74 of SEQ ID NO: 4;
viii. In the presence of terpene compounds presence of a mutated protein of the protein of SEQ ID NO: 5 or a homolog thereof preferably wherein the mutated protein of the protein of SEQ ID NO: 5 or a homolog thereof has a) a mutation at the position corresponding to the position 291 of SEQ ID NO: 5, and/or b) a mutation at the position corresponding to the position 274 of SEQ ID NO: 5 or thereafter wherein the mutated protein is shorter than the protein of SEQ ID NO:5 or the homolog thereof, or absence, inactivation or reduced abundance of the protein of SEQ ID NO: 5;
ix. Presence of a mutated protein of the protein of SEQ ID NO: 6 or a homolog thereof in the presence of terpene compounds, wherein the mutated protein of the protein of SEQ ID NO: 6 or a homolog thereof has a mutation at the position corresponding to the position 96 of SEQ ID NO: 6 (preferably mutation is a mutation replacing a Valine with Glutamic acid) and/or a mutation at the position corresponding to the position 67 of SEQ ID NO: 6, preferably replacing a Glycine with a Serine;
x. Absence, inactivation or reduced abundance of the protein of SEQ ID NO: 6 or a homolog thereof in the presence of terpene compounds;
xi. Modified protein of SEQ ID NO: 8 or a homolog thereof, preferably absence, inactivation or reduced abundance of the protein of SEQ ID NO: 8 or a homolog thereof, in the presence of terpene compounds;
xii. Modified protein of SEQ ID NO: 9 or a homolog thereof, preferably absence, inactivation or reduced abundance of the protein of SEQ ID NO 9 or a homolog thereof in the presence of terpene compounds;
xiii. Modified protein of SEQ ID NO: 7 or a homolog thereof, preferably absence, inactivation, increased activity or reduced abundance of the protein of SEQ ID NO 7 or a homolog thereof in the presence of terpene compounds;
xiv. any combination of the previous I to xiii;
   wherein the tolerance is improved compared to a non-modified organism

Preferably, the modified organism is employed in methods for the production of terpene esters, preferably monoterpene esters, from terpene compounds, preferably monoterpene alcohols.

A modified organism according to the invention may be produced based on traditional methods for mutating organisms and/or standard genetic and molecular biology techniques that are generally known in the art, e.g., as described in Sambrook, J., and Russell, D. W. “Molecular Cloning: A Laboratory Manual” 3d ed., Cold Spring Harbor Laboratory Press, Cold Spring Harbor, N.Y., (2001); and F. M. Ausubel et al, eds., “Current protocols in molecular biology”, John Wiley and Sons, Inc., New York (1987), and later supplements thereto, and also including technologies like CRISPR/CAS and the like.

The modified organism can be any cell selected from a bacterial cell, a yeast cell, a fungal cell, an algal cell or a cyanobacterial cell, a non-human animal cell or a mammalian cell, or a plant cell.

Specifically, the modified organism can be selected from any one of the following organisms:

### Bacteria

The bacterial modified organism can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus or Lactococcus. gram positive: like Bacillus, Streptomyces

Useful gram positive bacterial modified organisms include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus. Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

gram negative: E. coli, Pseudomonas, Rhodobacter, Paracoccus

Preferred gram negative bacteria are Escherichia coli, Pseudomonas sp., preferably, Pseudomonas purrocinia (ATCC 15958) or Pseudomonas fluorescens (NRRL B-11), Rhodobacter capsulatus or Rhodobacter sphaeroides, Paracoccus carotinifaciens or Paracoccus zeaxanthinifaciens).

### Fungi

### Aspergillus, Fusarium, Trichoderma

The modified organism may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).

Further preferred fungi include strains belonging to the subdivision Zygomycotina, class Mycoraceae, e.g. Rhizopus or Mucor, in particular Mucor hiemalis.

Yeast Such as the Following May Also be Used in the Invention:

Pichia or Saccharomyces. The fungal modified organism may be a yeast cell. Yeast as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes

Eukaryotic modified organisms further include, without limitation, a non-human animal cell, a nonhuman mammal cell, an avian cell, reptilian cell, insect cell, or a plant cell.

In a preferred embodiment, the modified organism is a modified organism selected from:
a) a bacterial cell of the group of Gram negative bacteria, such as Rhodobacter (e.g. Rhodobacter sphaeroides or Rhodobacter capsulatus), Paracoccus (e.g. P. carotinifaciens, P. zeaxanthinifaciens), Escherichia or Pseudomonas;
b) a bacterial cell selected from the group of Gram-positive bacteria, such as Bacillus, Corynebacterium, Brevibacterium, Amycolatopis;
c) a fungal cell selected from the group of Aspergillus, Blakeslea, Peniciliium, Phaffia (Xanthophyllomyces), Pichia, Saccharamoyces, Kluyveromyces, Yarrowia, and Hansenula;
d) a transgenic plant cell or a culture comprising transgenic plant cells, wherein the cell is of a transgenic plant selected from Arabidopsis spp., Nicotiana spp, Cichorum intybus, lacuca sativa, Mentha spp, Artemisia annua, tuber forming plants, oil crops, e.g. Brassica spp. or Brassica napus, flowering plants (angiosperms) which produce fruits such as but not limited to strawberry or raspberry plants and trees; or
e) a transgenic mushroom or culture comprising transgenic mushroom cells, wherein the microorganism is selected from Schizophyllum, Agaricus and Pleurotisi.

More preferred modified organisms from organisms are modified organisms from microorganisms belonging to the genus Escherichia, Saccharomyces, Pichia, Rhodobacter, Pseudomonas or Paracoccus, (e.g. Paracoccus carotinifaciens, Paracoccus zeaxanthinifaciens) and even more preferred those of the species E. coli, S. cerevisae, Rhodobacter sphaeroides, Rhodobacter capsulatus, or Amycolatopis sp.

Particularly preferred is a Rhodobacter modified organism selected from the group of Rhodobacter capsulatus and Rhodobacter sphaeroides, or a Escherichia coli.

A further aspect of the invention is to a mutated protein selected from the group of:
i. a mutated variant of the protein shown as SEQ ID NO: 2 or a homolog thereof wherein the protein in order of preference only the first 54, 53, 52, 51, 50, 49, 48 or 47 amino acids from the N-terminus with the protein of SEQ ID NO: 2 or homolog thereof of the non-modified organism.
ii. a mutated variant of the protein shown in SEQ ID NO: 2 or a homolog thereof whch has a mutation at the position corresponding to the position 48 of SEQ ID NO: 2;
iii. a mutated variant of the protein shown SEQ ID NO: 4 or a homolog thereof has a mutation at the position corresponding to the position 74 of SEQ ID NO: 4;
iv. a mutated variant of the protein of SEQ ID NO: 5 or a homolog thereof that has has a) a mutation at the position corresponding to the position 291 of SEQ ID NO: 5, and/or b) a mutation at the position corresponding to the position 274 of SEQ ID NO: 5 or thereafter wherein the mutated protein is shorter than the protein of SEQ ID NO:5 or the homolog thereof;
v. a mutated variant of the protein of SEQ ID NO: 6 or a homolog thereof that has a mutation at the position corresponding to the position 96 of SEQ ID NO: 6, preferably the mutation is a mutation replacing a Valine with Glutamic acid) and/or a mutation at the position corresponding to the position 67 of SEQ ID NO: 6, preferably replacing a Glycine with a Serine;

Further embodiments of the invention are to any nucleic acids encoding the mutated protein of the invention, to expression cassettes comprising a nucleic acid encoding the mutated protein of the invention, to a vector comprising a nucleic acid encoding the mutated protein of the invention, to a host cell comprising a nucleic acid encoding the mutated protein of the invention and to a recombinant non-human organism comprising a mutated protein of the invention.

In one preferred embodiment, the modified organism or the mutated protein of the invention is used in the production of one or more terpene compounds and/or one or more terpene esters. The inventive method for producing a terpene compound and/or a terpene ester, preferably comprises the following steps: (a) culturing a modified organism of the invention, under appropriate conditions, and (b) obtaining from the modified organism of step (a) the terpene compound and/or the terpene ester.

In another preferred embodiment, the modified organism is suitable for carrying out the methods of the invention.

For instance, the modified organism can be used in a method for preparing a monoterpene ester, comprising esterifying a monoterpene alcohol to a monoterpene ester, in the presence of an alcohol acyl transferase. To this end, the modified organism preferably heterologously expresses the desired alcohol acyl transferase. It is preferred that the monoterpene alcohol is linalool, geraniol, alpha terpineol, gamma terpineol, lavandulol, fenchol, perillyl alcohol, menthol or verbenol, and if production of monoterpene esters is desired, any of these or a mixture of these is used as substrate for the alcohol acyl transferase. The monoterpene alcohol substrate can be produced by the modified organism and/or added exogenously to the modified organism, preferably when the organism comprises one or alcohol acyl transferase suitable for the production of the monoterpene esters.

A further aspect of the present invention is a method for increasing the tolerance to one or more terpene compounds, of a modified organism compared to a non-modified organism, including the steps of creating the modified organism of the invention and optionally maintaining said modified organism.

In a preferred embodiment, the invention is a method for production of one or more terpene compounds using an organism, including the steps of creating the modified organism of the invention, maintaining said modified organism in the presence of terpene compounds under conditions suitable for the modified organism to grow and produce said one or more terpene compound and optionally separating the one or more terpene compounds from said modified organism.

In a preferred embodiment the methods and modified organisms of the invention are directed to the production of one or more terpene compounds wherein at least one terpene compound is a C4 and C5 alcohol.

The methods, the use or the modified organism of the invention wherein terpene compound has a log P value of 2.0 or less, preferably 1.5 or less and/or has a solubility in water under standard conditions of at least 1.0 g/l, preferably 1.5 g/I or more.

A preferred embodiment of the invention directed to the methods, the use, the mutated protein or the modified organism of the invention wherein the tolerance to isoprenol, prenol, butanol, isobutanol, Vanillin, Geraniol and/or Citral (preferably both Geranial and Neral), preferably to isoprenol, prenol, butanol, isobutanol and/or Vanillin, is increased compared to a non-modified organism.

A further embodiment is a method, use or modified organism of the invention, wherein the modified organism comprises a) a knock-out or a deletion in part or full of the gene encoding for the protein the protein of SEQ ID NO: 3 or a homolog thereof, a knock-out or b) a deletion in part or full of the gene encoding for the protein the protein of SEQ ID NO: 2 or a homolog thereof, or c) presence of a mutated protein of the protein of SEQ ID NO: 2 or a homolog thereof in the presence of terpene compounds, wherein the mutated protein of the protein of SEQ ID NO: 2 or a homolog thereof shares from the N-terminus only the first 50, 49, 48 and even more preferably the first 47 amino acids with the protein of SEQ ID NO: 2 or homolog thereof of the non-modified organism, or any combination of a) to c).

In yet another embodiment the method of any of the invention include the step of downregulating the expression of the gene encoding the protein of SEQ ID NO: 6 or a homolog thereof, deleting the gene encoding the protein of SEQ ID NO: 6 or a homolog thereof or knock out the gene encoding the protein of SEQ ID NO: 6 or a homolog thereof

In a preferred embodiment the invention is directed to method for increasing tolerance to Vanillin of a modified organism compared to a non-modified organism including the step of in a modified organism expressing of or generating a DNA sequence encoding a protein that shares in order of preference only the first 54, 53, 52, 51, 50, 49, 48 or 47 amino acids with the protein of SEQ ID NO: 2, wherein the modified organism has the further characteristic that the proteins of SEQ ID NOs: 1 and/or 2 or homologs thereof are absent, inactive or substantially reduced.

Further encompassed by the invention is the use of a deregulated protein of SEQ IDNO: 2 or a homolog thereof to increase growth of modified organisms in the presence of terpenes.

The mutated or deregulated protein of SEQ ID NO: 2, or homolog thereof, has in one preferred embodiment a mutation of the histidine residue corresponding to the position 48 of SEQ ID NO: 2 resulting in a frameshift, preferably a frameshift shortening the resulting protein compared to the protein of SEQ ID NO: 2.

In another preferred embodiment any of the sequences of SEQ ID NOs: 1 to 9 are mutated to carry the mutations as shown in table 3 for the respective protein.

Further, the invention includes the use of the modified organism or the mutated protein of the invention:
(i) for heterologous reconstitution of a terpene biosynthetic pathway;
(ii) for producing an industrial product, preferably a flavour or fragrance, a biofuel, a pesticide, an insect repellent or an antimicrobial;
(iii) for producing an aliphatic and/or aromatic monoterpene ester from a monoterpene alcohol, preferably from a tertiary monoterpene alcohol.

The invention further pertains to the use of the modified organism or the mutated protein of the invention, the nucleic acid of the invention, the vector or gene construct of the invention, the host cell of the invention, or the transgenic non-human organism of the invention (i) for heterologous reconstitution of a terpene biosynthetic pathway; (ii) for producing an industrial product, preferably a flavour or fragrance, a biofuel, a pesticide, an insect repellent or an antimicrobial; (iii) for producing an aliphatic and/or aromatic monoterpene ester from a monoterpene alcohol, preferably from a tertiary monoterpene alcohol; (iv) for detoxifying a monoterpene alcohol in fermentation, thereby increasing monoterpene production by said fermentation.

The invention also concerns the use of the modified organism or the mutated protein of the invention, the nucleic acid of the invention, the vector or gene construct of the invention, the host cell of the invention, or the transgenic non-human organism of the invention.
(i) for heterologous reconstitution of a terpene biosynthetic pathway;
(ii) for producing an industrial product, preferably a flavour or fragrance, a biofuel, a pesticide, an insect repellent or an antimicrobial;
(iii) for producing an aliphatic and/or aromatic monoterpene ester from a monoterpene alcohol, preferably from a tertiary monoterpene alcohol;
(iv) for detoxifying a monoterpene alcohol in mixture of microorganisms such as the modified organisms of the invention and bacteria or fungi (e.g. yeast), thereby increasing monoterpene production by said mixture of microorganism.

Preferred tertiary monoterpene alcohols include, but are not limited to, linalool (S-linalool and/or R-linalool), alpha terpineol, fenchol, gamma terpineol, p-cymene-8-ol, p-menth-3-en-1-ol, p-menth-8-en-1-ol, 4-carvomenthol, 4-Thujanol.

One aspect of the invention are methods for the production of monoterpene esters by production of the monoterpenes according to the methods of the inventions and the modified organisms of the invention, and esterifying these to monoterpene esters. Such esterification may be done in parallel, e.g. within the same modified organism of improved production potential for monoterpenes according to the invention, or in a subsequent step using the same or different cells or esterification enzymes either in an extract or isolated, or chemical esterification, preferably after isolation and purification of the monoterpenes. The monoterpene ester produced in accordance with this method of the invention may be used as such, e.g. as a flavour or fragrance, as an insect repellent, as a pesticide, or as an antimicrobial; it can also be used for producing biofuel, or may be used as a starting material for another compound, e.g. another flavour or fragrance.

### DESCRIPTION OF FIGURES

FIG. 1 depicts the structural formulas of the following substances: A—Isoprenol (3-methyl-3-buten-1-ol), B—Isobutanol, C—Prenol, D—Geraniol and E—Vanillin

FIG. 2: Growth of isolated strains at the Isobutanol concentration where growth is 50% inhibited (EC50) (65 mM).

FIG. 3: Growth of isolated strains at the Prenol concentration where growth is 50% inhibited (EC50) (40 mM).

FIG. 4: Overview of occurrence of mutations during the evolution experiment.

A Total frequency of mutations and time-point of first occurrence in the cultures of mutations.

B: Persistence of mutations in adapted strains. Persistence is the frequency of the mutation corrected for the time-point of occurrence in the evolution experiment.

FIG. 5:

A: Hypothetical regulatory elements in the yghB promoter region. The upper strand and below the complementary sequence are shown. The black rectangle marks the start of the open reading frame (ORF) and the starting Methionine (Met). Upstream of this the untranslated region (UTR) is shown. In this area a possible regulatory motif upstream of −35 region, a direct repeat and inverted repeat downstream of −35 region are predicted. Black arrows are shown for a motif in the region of the deletion, which is marked by a checkered box, and the inverted motif. Transcription factor binding could possibly inhibit transcription acting as a repressor.

B: Consensus-sequence of hypothetical binding motif (van Helden, Andrë and Collado-Vides, 1998).

C: Close-up of promoter part shown in A: The deletion (dark grey bar) upstream of yghB ORF and annotation of putative regulatory motif (grey arrows) in more detail.

D: Sequence changes in promoter region of yghB in the adapted strains. The upper strands represent the wild-type sequence (P_yghB wt)), and the lower strands the sequence with the deletion (P_yghB del). The checkered box represents the deletion that has changed the wildtype promoter sequence to the one shown at the bottom. The black rectangle marks the start of the open reading frame (ORF) and the starting Methionine (Met). Upstream of this the UTR is shown.

FIG. 6: Significant differentially expressed transcripts compared to wild-type. Genes that were significantly differentially expressed (P<0.05) in all three biological samples for the different DEalgorithms. (A) Significantly overexpressed transcripts (log 2>1.35) (B) Significantly downregulated transcripts (log 2<−2.7).

FIG. 7: Relative fitness (μₛₜᵣₐᵢₙ/μ_{wt}) of mutant rob H48fs expressing strains at 50 mM Isoprenol.

FIG. 8: Relative fitness (μₛₜᵣₐᵢₙ/μ_{wt}) of combinatorial knock-out strain of rob and marC expressing mutated robH48fs with 0 μM IPTG induction at 50 mM Isoprenol.

FIG. 9:

A: Shows the results of the screen for Butanol toxicity at different Butanol concentrations and growth rates of the original strain at various concentrations and of an adapted strain at 7.5 g/L. The abbreviation Mut T6 A defines the mutated strain of the T6 generation of isolate A as described herein above.

B: Evaluation of growth rate of different engineered strains with 5 g/L Butanol. Wild-type growth rate in this assay was 0.25 1/h. yghB and rob H48fs were expressed from the leaky IPTG-inducible promoter without induction.

FIG. 10:

A: Evaluation of Vanillin tolerance. E. coli wild-type strain was grown at different Vanillin concentrations. At 1 g/L Vanillin also the growth rate of an adapted strain was tested. Mut T6 A defines the mutated strain of the T6 generation of isolate A as described herein above

B: Evaluation of growth rate of different engineered strains with 1.5 g/L Vanillin. Wild-type growth rate in this assay was 0.23 1/h. yghB and robH were expressed from the leaky IPTG-inducible promoter without induction.

FIG. 11:

Relative fitness (μₛₜᵣₐᵢₙ/μ_{wt}) of ΔrraA at 50 mM Isoprenol in BW25113 strain.

### EXAMPLES

1. Results

1.1 Investigation of mode of adaptation in adapted strains

At the end of the adaptive evolution against Isoprenol several strains exhibiting an increased tolerance towards Isoprenol were isolated. The tolerance against Isoprenol was confirmed with the established toxicity assay of growth in M9 medium with Isoprenol in baffled sealed 250 mL flasks. To investigate the mode of action of the tolerance trait, chemicals with similar properties were tested.

Since often chemicals with solvent-like properties interfere with membrane function a standard assay, Propidium Iodide staining, was used to characterize the cell membrane properties under Isoprenol stress in the evolutionary adapted strains.

### 1.1.1 Tolerance Towards Different Chemicals

To assess the limitations of the tolerance mechanism, tolerance against three additional chemicals was tested. We tested the biological isomer of Isoprenol, Prenol, the branched alcohol Isobutanol and the monoterpene Geraniol.

### Table A: Characteristics of Some Terpenes

| Isoprenol | | Isobutanol | |
|---|---|---|---|
| logp = | 0.89 | logP = | 0.8 |
| Solubility = | 90 g/L | Solubility = | 85 g/L |
| Molecular Weight = | 86.13 g/mol | Molecular Weight = | 74.12 g/mol |

| Prenol | | Geraniol | |
|---|---|---|---|
| logP = | 0.91 | logP = | 2.5 |
| Solubility = | 170 g/L | Solubility = | 686 mg/L |
| Molecular Weight = | 186.13 g/mo | Molecular Weight = | 154.25 g/mol |

After establishing the half maximal inhibitory concentration of approx. 65 mM we tested the tolerance of Isoprenol against the final adapted strains that were also used for sequencing. All strains exhibit an increased tolerance against Isobutanol. The tolerance mechanism is not limited to Isoprenol but also Isobutanol is tolerated well. Of the adapted strains, the strain isolated from culture A exhibits the highest tolerance, whereas the strain isolated from culture C exhibits a smaller increase in growth rate. Isobutanol is very similar in its physicochemical properties, i.e. it has a similar log P value and solubility in water.

In a next set of experiments, we systematically determined the Prenol tolerance of the wild-type strain and found the half-maximal Prenol concentration to be at 40 mM. Despite the structural similarities between Prenol and Isoprenol, only strain isolate A exhibited an increased tolerance towards Prenol. Isolate C did not differ from wild-type tolerance and strain isolate B even had a decreased growth rate at 40 mM Prenol.

The differential tolerance of the different strain isolated might hint at different genotypes despite the same Isoprenol-tolerant phenotype.

Finally, the monoterpene compound Geraniol was tested. All of the isolated adapted strains displayed a highly elevated susceptibility against Geraniol. Not only can the trait increasing Isoprenol tolerance not protect from Geraniol toxicity, but the mechanism seems to further toxify the compound. Since in the adapted strains we found no evidence for Isoprenol degradation, it seems unlikely that Geraniol is increasingly degraded by those strains, so that a toxic degradation product could accumulate. Rather the tolerance mechanism must change the structure of the cell components in such a way, that those components are more susceptible to the toxic effect of Geraniol.

### 1.1.2 Membrane Permeability of Adapted Strains

With a log P close to 1 Isoprenol might exert its toxic effect by increasing the membrane permeability (Heipieper et al., 1994). To test this, Propidium Iodide (PI) staining was used. Propidium Iodide staining is a dead/live staining, since dead cells usually have defective cell-membranes, the staining can traverse the membrane and intercalate in the cell's DNA. This means that Propidium Iodide staining is suitable to detect cell-membrane damage.

Untreated wild-type cells show a median PI mediated fluorescence of approx. 1.4*10³, the median fluorescence increases 100-fold if the cells are treated with the disinfectant Bacillol AF prior to staining which is used as a positive control of the staining procedure. E. coli cells that were incubated with 50 mM Isoprenol, i.e. an intermediate Isoprenol concentration where cells still grow, have a median PI intensity of approx. 1.3*104 located between the intensities of live and Bacillol treated dead cells. Since this population is still actively growing, this means that cellmembrane integrity is indeed compromised by Isoprenol, but not to such an extent as to abolish growth.

It is interesting to note, that Isoprenol treatment results in a monomodal shift to higher PI staining. Isoprenol could in principle also increase the killing of alive bacteria, which would have resulted in a bimodal split of Isoprenol treated cells in ‘alive’ and ‘dead’ according to the staining. This is another indication that Isoprenol destabilizes the cell-membrane.

Next, we investigated how the adapted strains would react to Isoprenol treatment. Isolate A to C had a decreased median of 2.2 to 3.4*10³ PI fluorescence intensity compared to the wild-type Isoprenol treated cells. However, the PI-fluorescence remained slightly increased compared to wild-type untreated cells. This means that evolutionary adapted cells have developed a mechanism to cope with the membrane stress and in part restore membrane integrity, thus reducing the permeability for the PI staining.

### 1.2 DNA-Sequencing of Target Strains

To untangle the genetic basis of the observed adaptive mechanism, i.e. the tolerance towards Isoprenol, Isobutanol and Prenol and the decreased membrane permeability under Isoprenol stress, several strains were isolated from the adaptive evolution experiment and sequenced. As listed in Table 1 strains were isolated after 32 to 226 generations ranging from Isoprenol concentrations from 64 to 80 mM. Cryo-cultures of each of the three evolutionary cultures were streaked out on LB agar containing Isoprenol, the 5 largest strains were subsequently assessed in their growth in M9 with Isoprenol and the fastest culture was preserved and used for sequencing. In addition to the adapted strains, one wild-type culture was prepared for sequencing.

TABLE 1
Generations and Isoprenol concentration of isolated cultures

| | Generations | Isoprenol [mM] |
|---|---|---|
| T1 | 32 | 64 |
| T2 | 62 | 72 |
| T3 | 108 | 80 |
| T4 | 126 | 80 |
| T5 | 149 | 80 |
| T6 | 177 | 80 |
| T7 | 226 | 80 |

### 1.2.1 Mutations Identified in the Evolution Experiment

The mutations that were identified in the experiment are listed in Table 2 E. coli MG 1655 wildtype strains occur in different variants (Freddolino, Amini and Tavazoie, 2012). Our wild-type variant has a reconstituted gatC gene, which is part of the galactitol PTS, and a functional gIrR glycerol 3-phosphate repressor. In addition, there is a variation in the repeat REP321j.

TABLE 2
Mutations that occurred in the strains isolated from the evolution experiment

| Gene | Mutation | Freq | Time | Description |
|---|---|---|---|---|
| — | IS1 non-coding G->A | 5% | T5 | |
| frmR | V(86)-> G | 5% | T7 | Formaldehyde repressor |
| frmR | Q(21) -> frameshift | 5% | T7 | |
| gltA | E(116) ->K | 9% | T4 | Citrate synthase |
| plsX | Q(274)KS -> QRA | 9% | T3 | Fatty acid/phospholipid synthesis protein |
| | STOP | | | |
| plsX | G(291) -> C | 9% | T4 | |
| fabF | F(74) -> C | 45% | T3 | Component of 3-oxoacyl-ACP synthase II |
| fabF | wt | 5% | T4 | |
| marC | stop -> frameshift | 9% | T4 | Inner membrane protein involved in |
| marC | 1(135) -> stop | 9% | T2 | multiple antibiotic resistance |
| marC | M(35) -> stop | 45% | T3 | |
| gate | G(306) -> frameshift | 100% | T0 | PTS system galactitol-specific El IC |
| | | | | component |
| yffS | A ->A (silent) | 5% | T3 | CPZ-55 prophage; uncharacterized |
| | | | | protein |
| yfgO | A(154) -> V | 5% | T2 | Function unknown, predicted membrane |
| | | | | permease |
| iscR | H(107) -> L | 9% | T5 | Iron-sulfur cluster Regulator |
| srmB | D(157) -> N | 9% | T2 | SrmB is a DEAD-box protein with RNA |
| | | | | helicase activity that facilitates an |
| | | | | early step in the assembly of the 50S |
| | | | | subunit of the ribosome |
| P_{yghB} | | 32% | T4 | Required, with yqjA, for membrane integrity |
| glpR | A(51) -> frameshift | 100% | T0 | The sn-Glycerol-3-phosphate repressor,” |
| | | | | GlpR, acts as the repressor |
| | | | | of the glycerol-3-phosphate regulon |
| | | | | (Gain of Function Mutation?) |
| trkH | G(156) -> D | 9% | T5 | TrkH is a potassium ion transporter |
| rraA | V(96) -> E | 9% | T4 | RraA inhibits ribonuclease E activity |
| rraA | G(67) -> S | 5% | T4 | by binding to and masking the C-terminal |
| | | | | RNA binding domain of RNase E. |
| plsB | Q(322) -> R | 5% | T2 | Membrane-bound glycerol-3-phosphate |
| | | | | acyltransferase catalyzes the |
| | | | | first committed step in phospholipid biosynthesis |
| REP321j | | 100% | T0 | |
| rob | G(273) -> stop | 5% | T5 | transcriptional regulator |
| rob | Y(103) -> stop | 9% | T6 | implied in solvent tolerance |
| rob | H(48) -> frameshift | 18% | T6 | |
| creC | L (191) -> W | 5% | T5 | CreC is a carbon source responsive |
| | | | | sensor kinase |

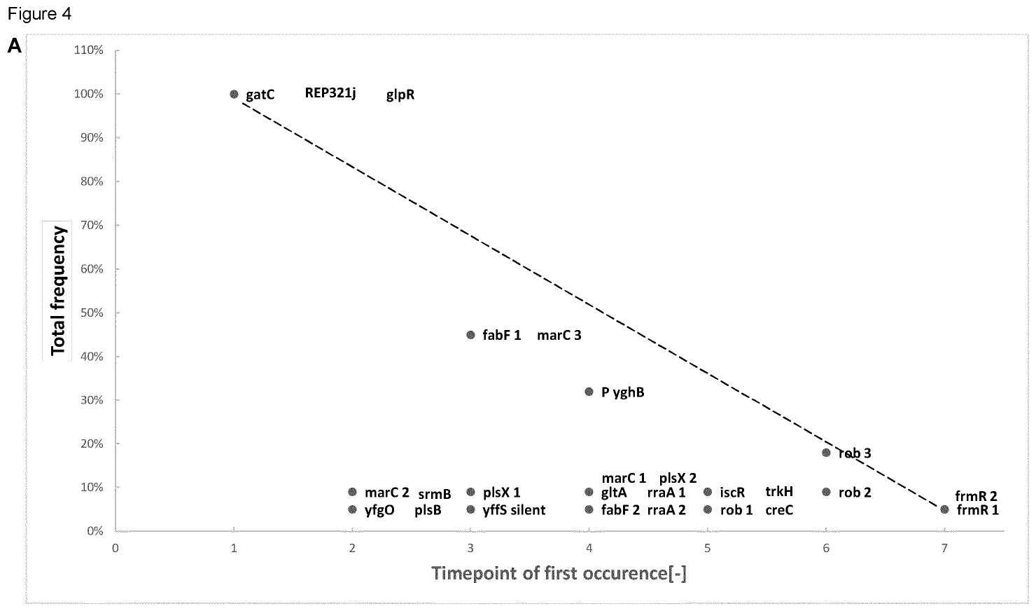

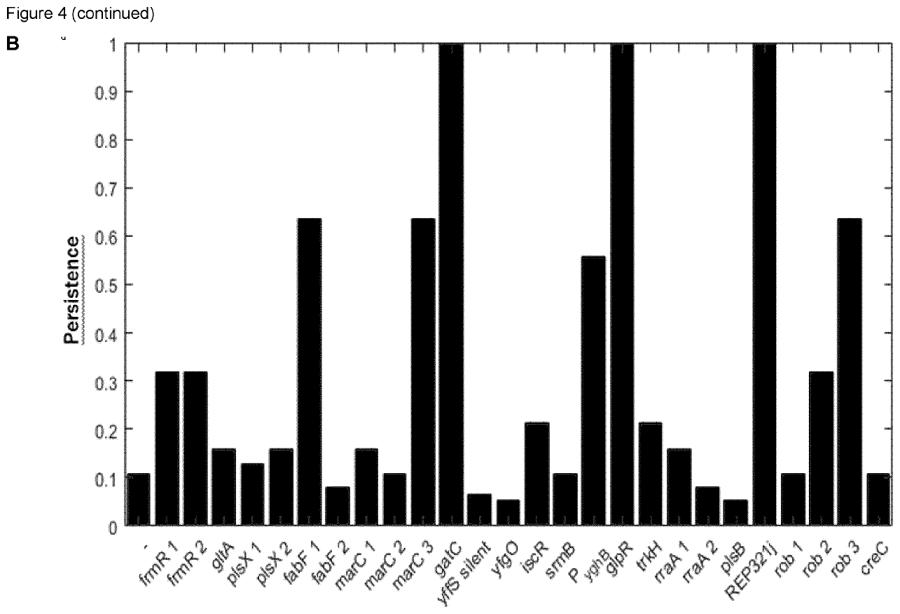

To give an overview about the time-course of mutation acquisition and their location in the genomes the results are schematically presented in FIG. 4A. It can be appreciated that from the beginning of the experiment the number of mutations steadily increases, however there also are mutations that appear but are lost again. The mutations do not seem to be limited to specific loci but are spread throughout the genome.

Most mutations are present at a relatively low frequency compared to all sequenced genomes of less than 10% (FIG. 4). There are four mutations that are present at a higher frequency.

This becomes apparent if one calculates the ‘persistence’ of each mutation, i.e. the frequency normalized to the number of remaining time-points in the evolution experiment. This means, if a mutation occurs in the beginning and remains in all culture, persistence will be 100%. If a mutation occurs in the middle of the experiment, but is not lost, the total frequency would be 50% but the persistence will be 100%. Consequently, the variations identified in the wild-type have a persistence of 100% but those genes can be excluded from the analysis. The four mutations with a high persistence are fabF F74C, marC M35stop, P_{yghb}Δ-35 and rob H48frameshift.

1.2.2 fabF F74C and marC

The highly persistent mutation fabF F74C has already been described in previous mutation experiments screening for 1-butanol (Haeyoung and Jihee, 2010). FabF encodes β-ketoacyl-ACP synthase II and is part of the fatty acid biosynthesis. This mutation increases the concentration of cis-vaccenic acid compared to wild-type FabF activity.

A disrupted version of marC has previously been identified in an adaptive evolution experiment of E. coli EcNR1 against Isobutanol (Minty et al., 2011). marC is a conserved membrane protein, deletion of the protein yielded an Isobutanol tolerant phenotype. The most frequent mutation in the marC gene present in our evolutionary experiment is the introduction of a stop-codon after M35, this leaves only approx. 15% of the native protein. It is likely that this mutation abolishes the function of the marC gene, however the truncated version might still have tolerance-benefit.

### 1.2.3 Rob

The next highly significant targets are mutations in the rob gene. The rob gene is a constitutively expressed regulator and its regulon is shared with the marA/soxS regulators (Rosenberg et al., 2003; Griffith et al., 2009). The regulon is involved in antibiotic resistance, superoxide resistance and tolerance to organic solvents (Aono, 1998). Overexpression of rob confers tolerance to Cyclohexane and n-Hexane, deletion makes it susceptible to those compounds (White et al., 1997). Two mutations in our sequencing results introduce premature stop codons after G273 and Y103, the most prevalent mutation introduces a frameshift after H48. The H48 frameshift mutation disrupts the protein in its Helix-turn-Helix domain (Source: https://www.rcsb.org/pdb/protein/P0ACI0), i.e. the part where the protein interacts with its DNA-binding site, thus rendering it possibly inactive.

### 1.2.4 P_{yghb} Δ-35

The last mutation with a high frequency is a mutation in the intergenic region between metC and yghB. metC belongs to the methionine biosynthesis pathway, yghB is a trans-membrane protein involved in temperature and antibiotic tolerance (Kumar and Doerrler, 2014). yghB belongs to the DedA-protein family in E. coli, double deletion of yghB and yaA (also belonging to the DedA-family) results in temperature sensitivity but can be restored by overexpression of mdfA an Na⁺—K⁺/H⁺ antiporter.

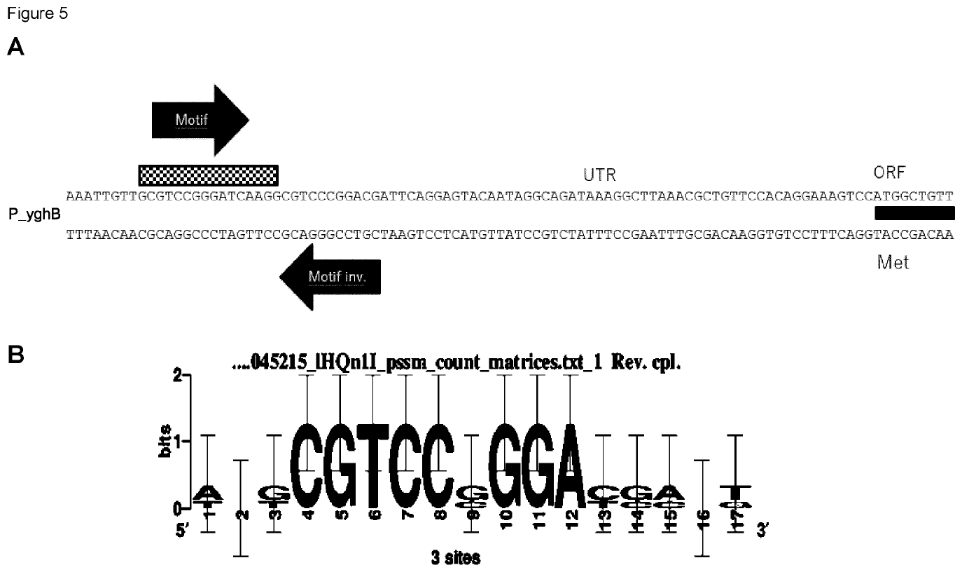

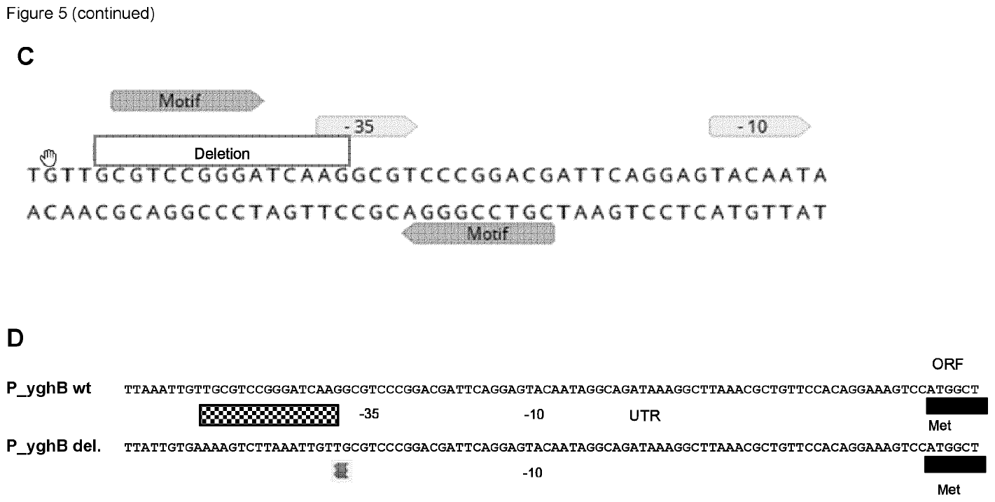

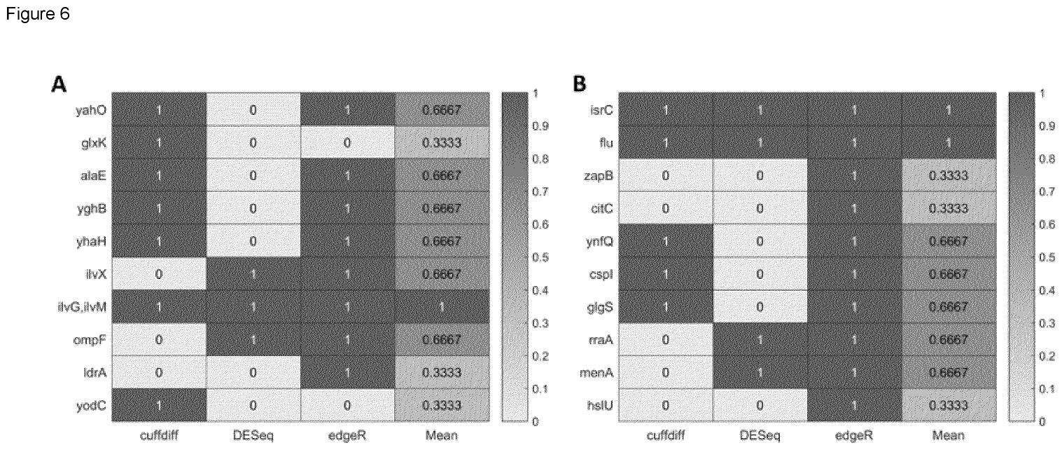

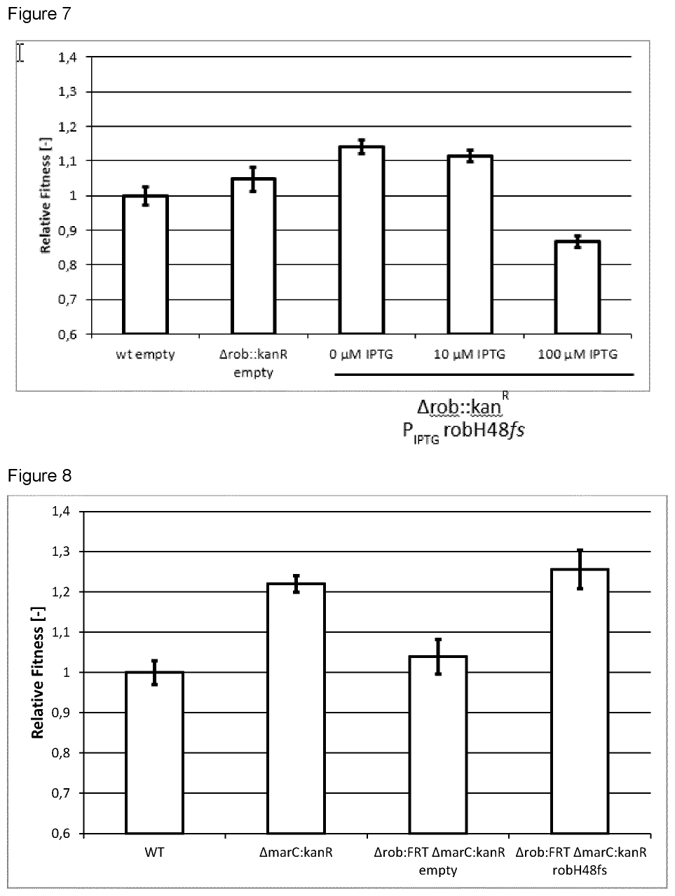

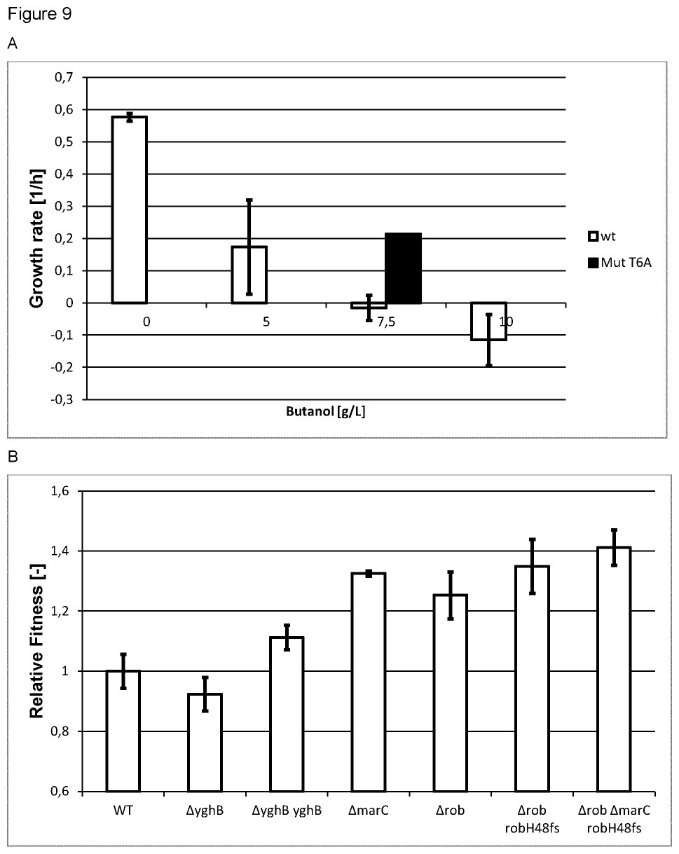

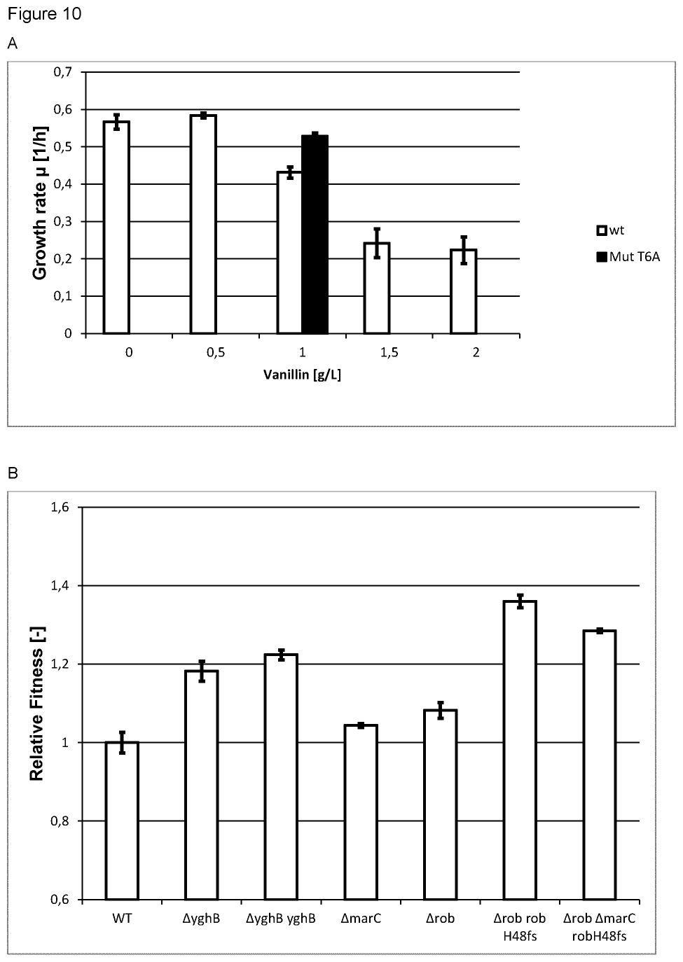

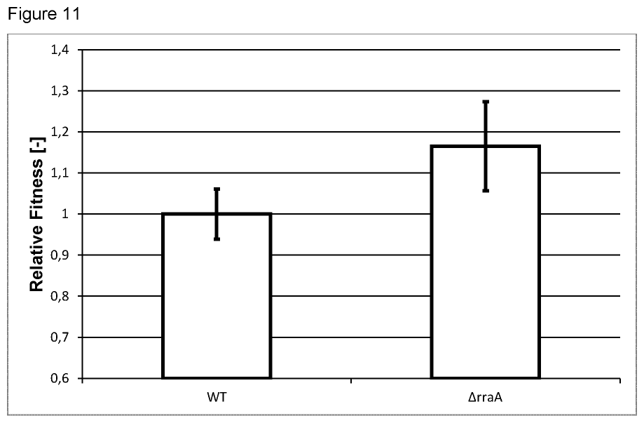

So far no regulators of yghB are known, however computational evidence suggests it is regulated by the σ⁷⁰ housekeeping sigma factor. Sequence analysis of the mutation reveals that a portion upstream of the −35 position in the wild-type is lost due to the deletion. This could delete a binding-site for a repressive promoter thereby deregulating expression of the yghB gene and possibly increasing yghB-mRNA concentration. See FIG. 5C

### 1.2.5 Genotype-Correlations

All other mutations have a much lower persistence in the experiment, however some target genes appear to have a higher frequency, e.g. the pIsX gene. If mutations occur in the same gene in different samples, it can be assumed that they might have the same phenotypic effect and therefore the same effect on gene functioning. To identify gene-sets for genotypes and how gene-targets are correlated we simplified the dataset considering only target-genes and not distinguishing between different target-gene mutations and performed a principle component analysis.

A principal component analysis (PCA-analysis) was conducted. The highest impact on the first and most important loading vector are the already identified targets fabF, rob, P_{yg}hb and marC. The second component defines a genotype consisting of pIsX, rraA and gItA. As expected, the phenotype at the end of the experiment (T7) is dominated by the first component

### 1.2.6 PCA-Component 2 Genotype

Interestingly the second genotype component is strongly present in culture A at T4 and T5. This genotype consists of mutations in the pIsX gene, which is part of the phospholipid biosynthesis pathway, a ribonuclease inhibitor rraA and the citrate synthase gItA. The mutations in pIsX gene might be a similar adaptation as the fabF mutation altering the fatty-acid composition of the cell. pIsX does not belong to the canonical phospholipid-pathway but has homology to an alternative route present in S. aureus (Yao and Rock, 2013). Supposing the alternative and the canonical pathway have different preferences for different fatty acids, this mutation might change the fatty-acid composition of the cell-membrane.

The other two mutations in the genotype might correlate to more pleiotropic effects of Isoprenol on the cell, such as energy metabolism and protein synthesis. The mutation in gItA might influence the allosteric response of the citrate synthase to the inhibiting effect of NADH (Duckworth et al., 2013) thus deregulating the TCA-cycle and influencing energy metabolism. There also appear two independent mutations in the ribonuclease E inhibitor rraA. A loss of function in this gene, would have an effect on tRNA and rRNA processing, but also make mRNA more unstable. Indeed the V96E appears to be at a rather conserved residue (Monzingo et al., 2003).

### 1.2.7 Other Mutations

The two prevalent mutations in the third component trkH and iscR might be responses to the loss of ions due to membrane stress by Isoprenol (Heipieper et al., 1994). iscR is the Iron-sulfur-cluster regulator and this mutation might differentially regulate Iron-sulfur cluster biogenesis. Increased potassium import is a known adaptation in Pseudomonas putida P8 towards solvent stress (Heipieper et al., 1994) and a similar mechanism might be at play in the mutation in the potassium ion transporter (Cao et al., 2011).

Adding to mutations in fatty-acid metabolism genes in fabF and pIsX there is one further mutation found once in the plsB gene necessary for phospholipid biosynthesis. With yfgO there is also another membrane protein target of a mutation, in addition to marC and yghB.

Interestingly two of the last three strain-isolates (T7 B and C) exhibit independent mutations in the frmR repressor that regulates formaldehyde metabolism. Although formaldehyde susceptibility was tested in the previous report, and no difference between Isoprenol with high and low residual formaldehyde content was found, this might correspond to a long-term adaptation, where accumulation of formaldehyde becomes critical. Interestingly the strains with mutations in the frmR genes have a higher susceptibility to Prenol.

### 1.3 RNA-Sequencing Based Analysis of Isoprenol Stress Response

How the adapted strains respond to the Isoprenol stress is determined by their genotype, however due to physiological changes of the strains and combinations of regulatory responses secondary effects might arise that are not directly evident from the genotype. These secondary effects ultimately are non-trivial targets for strain-engineering. To identify them we use RNA-Sequencing of the three final adapted strains and compared the transcriptome of the adapted strains to the wild-type in response to Isoprenol-stress.

In this analysis three standard algorithms, cuffdiff, edgeR and DESeq2, for the identification of differential expression (DE) were used. The algorithms differ in four major points, first raw read data usually needs to be corrected; this correction is mainly due to varying sequencing depth in the replicates. Second point is the underlying statistical model for the counts, in case of cuffdiff a beta negative binomial is assumed, whereas edgeR and DESeq2 assume a negative binomial distribution. Then algorithms differ in how the parameters of the distributions are estimated. Parameters of each distribution cannot be estimated directly from one data-point, since this is usually sparse (e.g. three biological replicates per sample), but have to be inferred from the total data. Finally different significance tests can be utilized to identify DE.

### 1.3.1 Differential Expression of Adapted Strains Compared to Wild-Type

Since the genotypes of the three adapted strains are very similar, we reasoned that the most profound transcriptome changes should occur in all three strains.

The top ten up and down-regulated genes in all three used algorithms are shown in (FIG. 6).

Due to the nature of transcriptome data, down-regulation is more difficult to confirm, since this also relies on the quality of alignment and high coverage. In the up-regulated gene-set we observe strong overexpression of the yghB-transcript. This corresponds well with the genome data of the adapted strain, since the yghB promoter is deregulated due to a deletion in the −35 region. We find now significant changes in the gene upstream of the deletion metC.

Other targets from the up-regulation are the ala-ala peptide exporter alaE, the outer membrane porin ompF, the valine biosynthesis genes ilvG, ilvM and the yahO gene involved in UV and X-ray tolerance. Other highly expressed genes are only significant in one of the three algorithms and are thus not considered plausible targets.

### 1.3.2 Differential Response of Lipid Biosynthesis and Rob-Regulon

Since many of the mutations in the experiment are targeted at fatty-acid biosynthesis we wondered if differential regulation will be present in the corresponding pathways.

Overall fatty-acid synthesis genes are up-regulated compared to the wild-type, most prominently fabH, fabB and c/sB. Interestingly only psd catalyzing a crucial step in phospholipid-synthesis is downregulated. However only for psd this downregulation is significant for all cultures and all algorithms, fabB upregulation is significant only with the edgeR algorithm.

The sequencing data identified the rob-regulator as one of the four most important mutational targets. From the genetic data alone, it is unclear what the exact effect of the mutation will be, we hypothesize that the mutations have a deleterious effect, and since rob acts as an activator this will decrease expression of genes in the rob-regulon.

The results showed that except for acnA, aldA and fumC all genes belonging to the rob-regulon (as designated by ecocyc.org) are down-regulated compared to wild-type. This supports the hypothesis, that the observed rob-mutations have a deleterious effect and deletion of the activator leads to subsequent downregulation of regulon-genes. Since the rob-regulon overlaps with Sox and Mar regulon, genes that appear up-regulated might be under stronger control of the other regulators. The strongest downregulation occurs in the part of the AcrAB-ToIC multidrug efflux pump small protein acrZ and in inaA, an acid inducible protein.

### 1.3.3 Differential Expression Between Adapted Strains

Finally, we wondered how the different mutants differed between each other in their transcriptome response to Isoprenol stress. As a comprehensive method to spot differences in all three datasets at once we performed a PCA-analysis of the differential expression data of each isolated strain compared to wild-type. This analysis shows that consistent among all three algorithms is the differential expression of frmRAB in the three mutant strains. As shown above, only the mutant strains 7B and 7C have a mutation in the frmR regulator that correlates with increased Prenol sensitivity. As an example, for the differential expression in the three isolates strains, between-strain differential expression as calculated by the edgeR algorithm was investigated. Indeed, the expression of frmRAB does not differ between strain B and C, however compared to strain A frmRAB is upregulated in strain B and C. This data suggests that both mutations in frmR have the same effect, i.e. a deregulation of the frmRAB operon resulting in a constitutive expression or up-regulation compared to wild-type and strain A.

### 1.4 Reconstitution of Mutations

### 1.4.1 Keio Knock-Out Strains

We began the investigation of the mutations discovered in the final phenotype by testing the knock-out strains of the most promising gene-targets from the readily available Keio collection. The Keio collection is implemented in the BW25113 background which we subsequently used as reference for tolerance testing when using strains derived from the Keio collection. Compared to MG1655 the BW25113 strain is auxotroph for arabinose and rhamnose. Since glucose is the sole carbon source in our growth-assays this should not impact the physiology of tolerance.

The wild-type BW25113 appears to have a slightly higher growth rate under Isoprenol stress then MG1655, however this difference is not significant. A knock-out of the regulator rob slightly increases the growth rate, but this difference is not significant. The Keio strain with deleted marC significantly increases the growth rate. This agrees with results obtained in a previous study on Isobutanol stress (Minty et al., 2011). We hypothesized that the mutation found upstream of the yghB gene increases gene expression, conversely deletion of the gene should have a negative effect on tolerance. Indeed, we observe a decreased growth rate under Isoprenol stress in the yghB deletion strain of the Keio collection.

1.4.2 yghB Reconstitution

To increase the tolerance to terpene by increasing the expression of yghB, an expression vector for yghB was constructed by Gibson cloning. The plasmid has a pUC derived ori, i.e. is a high copy plasmid (Hoschek, BQhler and Schmid, 2017). Expression is regulated by the P_{trc1O} promoter which is derived from the high expression trp promoter and the lacUV5 promoter and contains one lac1O operator for lac/expression (Brosius, Erfle and Storella, 1985). To control transcription, the plasmid harbors a copy of the lac/inhibitor. The expression plasmid was verified by colony-PCR and sequencing. In the host, it can be selected via ampicillin or chloramphenicol resistance and the plasmid contains an IPTG inducible Ptrc1O promoter used for expression of yghB.

yghB Overexpression in Wild-Type Background

As described in the previous report yghB mRNA levels are upregulated approximately 14-fold, therefore we hypothesized that additional expression of yghB from the overexpression plasmid in the MG1655 wild-type might yield mutant-like expression levels of yghB and restore the tolerance phenotype. It was found that full induction with 100 μM IPTG decreases the growth compared to an empty-vector control strain. The yghB overexpression strain without induction or low induction of 10 μM IPTG shows a small but insignificant increase in fitness.

1.4.2.1 yghB Overexpression in marC Knock-Out Background

Strong overexpression of yghB in the wild-type background did not have a positive fitness benefit. In the mutant strains of the evolution experiment the yghB mutation does not occur isolated but in conjunction with other mutations. Since single mutations might have a negative fitness effect and only with other mutations exert a positive fitness effect (Minty et al., 2011) we wanted to test yghB overexpression in the context of the mutation with the strongest fitness effect so far, i.e. the marC knock out. To this end we introduced the yghB expression plasmid in the ΔmarC strain of the Keio collection. Although the marC knock-out exhibits a significant fitness increase this does not affect the fitness effect of yghB induction. Minimal induction with 10 μM IPTG decreases the fitness slightly compared to the ΔmarC strain, strong induction with 100 μM IPTG decreases fitness of the marC-knock out to such an extent that it is below the wild-type strain. So far our data indicates that yghB overexpression has only negative effects on fitness. We wondered if our expression plasmid produces functional YghB and is able to complement a yghB knock-out strain.

1.4.2.2 yghB Overexpression in yghB Knock-Out Background

Finally, the yghB overexpression plasmid was transformed into the ΔyghB strain of the Keio collection. Knock-out of yghB decreases the fitness about 30%. The complemented knock-out strain with the yghB overexpression plasmid shows diverse responses to Isoprenol stress. Without induction fitness of the complemented strain slightly exceeds wild-type fitness, however this fitness increase is not significant. Mild induction of expression between 3 and 10 μM IPTG leads to an Isoprenol tolerance similar to the reference strain. Similar to previous experiments strong induction of 50 μM decreases tolerance again. The yghB plasmid is able to complement a yghB deficient strain, although only in a narrow induction regime.

Hypothetical Basis of yghB Dysregulation

During the planning of the CRISPR gRNA construct we observed that the deletion part in the promoter upstream of the −35 region contains a sequence motif that is directly repeated (with 1 bp exchange) overlapping with the −35 region and perfectly repeated on the opposite strand downstream of the −35 region (FIG. 5A). Using the TOMTOM tool of MEME Suite (Gupta et al., 2007) we identified fatty acid degradation regulator FadR and the cAMP receptor protein CRP as likely regulators with similar binding motifs, among prokaryotic motifs in general the Bacillus subtilis NatR regulator has the most similar binding motif.

1.4.3 Knock-Out Complementation with Mutant Proteins

Although we initially hypothesized that the mutations found in marC and rob gene likely lead to a loss of function it is unclear whether mutated proteins retain part of their function or have a different functionality and thereby a positive effect on Isoprenol tolerance. To test this we expressed the corresponding mutant proteins in the knock-out backgrounds.

1.4.3.1 marC

The most frequent mutation of the marC gene introduces a stop codon after the methionine at position 35 (M35stop) and thereby significantly truncates the protein after the first transmembrane domain. A plasmid for the expression of a version of marC with a stop after the methionine at position 35 was constructed using standard methods. The plasmid is based on a pUC background, can be selected via ampicillin or chloramphenicol resistance and contains an IPTG inducible Ptrc1O promoter used for expression of marC M35stop.

As described herein above a marC knock-out alone has an increased tolerance against Isoprenol. Complementation of the knock-out with an IPTG-inducible marC M35stop protein does not further increase tolerance against Isoprenol

1.4.3.2 rob

The transcriptional regulator rob is mutated by a frameshift at the histidine at position 48. This results in a truncated protein of 107 amino acids length. The protein binding HTH-motif might be intact; however the rest of the protein shares little similarity with the original protein. To test if such a frameshift version may have any effects when overexpressed in the knock-out background, a plasmid for the overexpression of robH48fs was constructed using standard methods. The plasmid is based on a pUC background, can be selected via ampicillin or chloramphenicol resistance and contains an IPTG inducible Ptrc1O promoter used for expression of rob H48fs.

Knock-out of the rob-gene results only in a minor increase of tolerance against Isoprenol. 8 shows that introduction of a plasmid containing rob H48fs further increases tolerance. This effect is lost again if the protein is strongly induced, possibly due to the additional metabolic burden of protein expression. The mutant Rob-protein might retain its DNA-binding capability due to an intact HTH-motif, however the regulatory function is possibly altered since the c-terminal interaction domain is missing.

### 1.4.4 Tolerance Testing of Double-Knock Outs

After examination of E. coli strains with one reconstituted mutation we wanted to examine possible epistatic effects of multiple gene mutations. To this end the kanamycin resistance cassette in the rob-knock out was replaced by FLP recombination. This facilitates the introduction of additional knock-outs with kanamycin resistance cassettes.

We tested the combinatorial effect of a double knock-out of rob with marC. Combination of both knock-outs resulted in a strain with an increased tolerance against Isoprenol, however the fitness increase compared to the wild-type strain was less than a single knock out of marC. It is also possible that a knock-out of rob alone does not reconstitute the actual mutation and, as shown above, the mutated Rob H48fs protein alters regulation to benefit Isoprenol tolerance. To test this, we introduced the plasmid expressing the mutated Rob H48fs protein into the double knock-out of rob and marC. Indeed, with the plasmid expressing the mutant protein the tolerance slightly increases compared to the marC kock-out alone.

### 1.5 Broad Applicability of the Increase in Tolerance in the Host Cells and the Methods of the Present Invention

### 1.5.1 Butanol

As another substance of toxic effects on microorganisms, butanol is known. Demonstrating the broad applicability of the host cells and the methods of the present invention the mutant strain exhibiting all 4 major mutations showed an increased tolerance at 7.5 g/L Butanol (the literature value for half-maximal concentration) (FIG. 9A).

Knowing that the experimental EC₅₀ in our experimental set-up is close to 5 g/L we subsequently tested the most relevant mutations at this concentration (FIG. 9B). Similarly, to Isoprenol tolerance, we find that knock-out of yghB decreases tolerance towards Butanol. Complementation of the yghB knock out with an yghB expression plasmid expressing yghB under leaky expression conditions (0 μM IPTG) increases relative fitness about 11%. As expected from the Isoprenol tolerance a knock-out of marC increases tolerance against Butanol 32%, interestingly also a rob knock-out elevates tolerance about 25%. The tolerance is further increased by leaky expression of rob H48frameshift in a Δrob background to 34%. The highest tolerance against Butanol with 41% increased growth rate can be observed with a double knock out of rob and marC complemented with rob H48fs expression.

### 1.5.2 Vanillin

Vanillin is a commercially interesting substance with some similarities to terpenes that also has negative effects on many microorganisms. To test the potential application of the tolerance mechanism towards this product we systematically evaluated the growth rate of wild-type E. coli MG1655 with Vanillin (FIG. 10A). In the concentration regime tested we did not find complete growth inhibition but only a reduction to ⅓ of wild-type growth rate. At an intermediate Vanillin concentration of 1 g/L an Isoprenol adapted mutant strain (Isolate A, in the 6^{th} generation MutT6A) shows a significantly increased growth rate.

For Vanillin half-maximal growth repression is achieved between 1 and 1.5 g/L. For reasons of comparison we tested significant mutations at a Vanillin concentration of 1.5 g/L (see FIG. 10B). In contrast to other tested chemicals an yghB knock-out has a positive effect on Vanillin tolerance and increases the growth rate 18%. Complementation of this strain with additional yghB expression has only a minor effect of additional 3% faster growth. Unexpectedly a knock-out of marC has no significant positive effect under Vanillin stress. Similarly, a rob knock-out only leads to a minor increase of 8%. If however the rob knock-out is complemented with the mutant version rob H48fs the tolerance increases to 36% compared to wild-type. Addition of the marC knock-out to this strain reduces the tolerance again to 28% increased growth rate.

1.6 Screening of Targets from RNA-Seq Experiment

Our RNA-Seq analysis of Isoprenol stress on adapted strains revealed a list of significantly up and down-regulated target genes in the adapted strains compared to wild-type. A down-regulated phenotype could in principle be mimicked by knock-out strains. To this end we tested strains of the Keio-knock out library towards their Isoprenol tolerance.

Of the 5 target genes glgS, rraA, menA, cspL and flu, only the rraA knock out displayed a significantly increased growth rate with 50 mM Isoprenol compared to the wild-type.

### 2 Discussion

### 2.1 Mutations Discovered in Adaptive Evolution Experiment

We initially identified a set of 22 mutations occurring during the course of the evolution. Of those 22, 4 target genes and mutations were highly stable and persistent from the time-point of occurrence in the evolution experiment. Literature research revealed that mutations of rob and yghB had not been implicated in solvent or alcohol tolerance before. The fabF mutation had been identified in Butanol tolerance previously (Jeong et al., 2012).

marC deletion mutants have been studied in the context of Isobutanol tolerance (Minty et al., 2011), however it was unclear whether truncated proteins such as MarC M35stop would have an additional tolerance effect. Our experiments revealed that expression of mutated MarC M35stop does not result in additional tolerance against Isoprenol. It is therefore likely that marC mutations act as gene deletions and our evolution experiment did not reveal a novel tolerance mechanism.

We also isolated three strains that contained mutations in the rraA gene, since RNA-Seq data showed a strong down-regulation of the rraA gene in the adapted strain it is possible that these mutations also have a deleterious effect on protein function. Testing revealed that indeed a rraA knock-out strain has an increased Isoprenol tolerance.

In another embdoiment the novel psXfmutants that appeared in 4 of the isolated strains are useful to increase the tolerance to said toxic substances such as terpenes in host cells and the methods of the present invention. A different pdsXmutant, PIsX E216G, has been discovered in Isobutanol tolerance evolution (Minty etaD., 2011), however its mechanism and effect are unclear.

TABLE 3
Major targets identified by genome-sequencing. The detailed mutation and its
frequency in the experiment is given in brackets. Effects of mutation on the cellular level
are provided, those listed in bold writing show the surprising findings of the current invention

| | | | Effect of mutation in the | |
|---|---|---|---|---|
| Gene | Mutation | Function | cell | Substances |
| yghB | Pyghb A-35 (0.325) | Membrane | Increased expression | Isoprenol |
| | | protein | (RNA-Seq data) | Butanol |
| | | | Dysregulation of native | Vanillin |
| | | | promoter | |
| rob | G273-Stop (0.05) | Transcription | Altered regulation by mutated | Isoprenol |
| | H48-fs (0.2) | factor | rob-protein H48fs | Butanol |
| | Y103-Stop (0.1) | | | Vanillin |
| marC | 1135-stop (0.1) | Membran | Deleterious effect | Isoprenol |
| | M35-stop (0.45) | Protein | | Butanol |
| | Stop-fs (0.I) | | | |
| fabF | F74-C (0.45) | Fatty-acid | Changes proportion of cis- | Isoprenol |
| | | synthesis | vaccenic acid | |
| plsX | G291-C (0.1) | Phospholipid | Effect unknown, alternative | Isoprenol |
| | Q274KS-QRA | synthesis | pathway | |
| | Stop (0.1) | | | |
| rraA | V96-E (0.09) | RNA-Stability | RNA-Seq downregulation | Isoprenol |
| | G67-S (0.05) | | Deleterious effect | |

2.2 yghB Promoter Mutation

The genome analysis revealed that upstream of the yghB gene, in close proximity to the −35 region, 15 bp are deleted in all final strain isolates. Additional investigation by RNA-Seq showed that in all adapted strains expression of the yghB gene is significantly upregulated 14-fold compared to the wild-type. Close inspection of the yghB promoter sequence showed that the −35 region might be flanked by two repeating sequence motifs. Interestingly the upstream repeat of the motif is deleted in the mutant strains. The architecture of this motif suggests a repressive effect of a possible DNA-binding factor. Deletion of the putative regulator binding site could lead to a deregulation of the promoter and thereby lead to increased mean expression of yghB. Since the motif has not been described in literature the role of the putative repressor remains unclear.

It could be the case, that yghB expression is repressed under Isoprenol stress in the wild-type and this repression is relieved in the mutant. However yghB is highly expressed in the wild-type; approx. 6 fold higher than median expression values. Another hypothesis could be that yghB expression is only heterogeneously repressed and that this heterogeneous repression of the subpopulation is relieved by the promoter deletion mutation. In this case it would be indicative to study the yghB promoter activity with fluorescence microscopy under Isoprenol stress.

The initial approach to reconstitute this mutation was to construct an overexpression plasmid. However expression of yghB in the wild-type background and strong induction only led to a reduced growth rate. If yghB was expressed without induction, i.e. relying on the leaky expression of the plasmid, in a ΔyghB background tolerance could be improved. This suggests that yghB has a non-linear limited effect on tolerance, i.e. yghB expression is only beneficial in a finetuned expression regime, and expression levels might exceed this regime if a high-copy plasmid with a strong expression promoter is used. Complementation of a ΔyghB with leaky yghB expression increased tolerance against Isoprenol, Butanol and Vanillin. However under Vanillin stress also a knock-out of yghB improved tolerance.

### 2.3 rob Mutation

In the isolated strains 3 different mutations of the rob gene were identified. Two mutations cause a truncation of the protein after G273 and Y103, the most prevalent mutation causes a frameshift after H48 and results in a 107 aa long protein. Deletion of the rob gene had only a modest effect on the tolerance against Isoprenol. Complementation of a rob knock out strain with the mutated rob H48 frameshift increases the tolerance against Isoprenol significantly. This knock-out strain with the rob H48 fs also results in high tolerance against Vanillin and Butanol. The mutated Rob H48fs contains part of the HTH DNA binding motif hence the protein can still bind to DNA but lost its ability to react to molecular cues with its C-terminal receiver domain (Griffith et al., 2009).

### 2.4 Combinatorial Effects

In the course of evolutionary processes acquisition of new mutations is often aided by so called epistatic effects, i.e. the fitness benefit of two mutations combined exceeds the sum (or product) of the single fitness benefits. We found for the combination of marC and rob knock-out with Rob H48fs expression an additional fitness benefit for Isoprenol and Butanol tolerance, however this combination did not show any synergistic effects. In the case of Vanillin toxicity, addition of the marC knock-out to the Arob rob H48fs strain decreased the fitness, which is evidence of negative epistatic interaction.

2.5 Knock-Out Targets from RNA-Seq

In our RNA-Seq experiments comparing the expression of adapted strains against the wild-type strain under Isoprenol stress we identified a set of highly up- and down-regulated genes in the adapted strains. If the differential regulation is beneficial for tolerance molecular engineering of up and down regulation could mimic this effect. This apparently is the case for the upregulation of yghB as shown above. Extreme down-regulation of a target gene in the adapted strains could theoretically be achieved by knock-out of the target genes.

To this end a set of available knock-out strains was tested towards their Isoprenol tolerance. We identified the rraA gene as a target knock-out that is beneficial for Isoprenol tolerance. In addition to the down-regulation in the mutant strains we also observed two mutations in early strain isolates of the evolution experiment. Since a knock-out strain achieves a positive tolerance effect we hypothesize that the amino acid exchanges in the mutant RraA protein, V96-E and G67-S, might have a negative effect on the in vivo RraA function. The G67-S mutation is adjacent to a structural β-sheet element but also present in other species. The more prevalent V96-E mutation is at a highly conserved valine residue and might be critical for RraA function (Monzingo et al., 2003). As an inhibitor of Rnase E the absence of rraA decreases pleiotropically the level of mRNA transcripts (Lee et al., 2003).

### 2.6 Expansion of Positive Mutations to Additional Chemicals

The host cells and the methods of the present invention achieve increased Isoprenol tolerance in microorganisms such as E. coli. Moreover, the host cells and the methods of the present invention increase the tolerance of microorganisms to additional chemicals. The host cells of the present inventions have a higher tolerance against Butanol and also against Isobutanol and applied to other alcohols or aldehydes with C4 and C5 bodies.

We also tested the adapted strains for their tolerance against the monoterpene compound Geraniol, however we found that those strains have an increased susceptibility against Geraniol. We reasoned that the present tolerance mechanisms only apply to compounds with similar physical and chemical properties, therefore we did not test Citral and Menthol which both have a lower solubility in water and a higher log P value than Geraniol. Consequently, we tried to determine the limit physical and chemical properties where the tolerance mechanism would work and chose Vanillin which has an intermediate log P value between Isoprenol and Geraniol. We found that Vanillin tolerance can be achieved by host cells and the methods of the present invention with the exception that marC did not play a role in Vanillin tolerance. Expression of mutated rob H48 fs in a Δrob background had a strongly positive effect on tolerance. yghB also plays a role in Vanillin tolerance, however in a different manner than for the C4 and C5 alcohols. Whereas a knock-out of yghB had a negative effect on Isoprenol and Butanol tolerance it has a positive effect in Vanillin tolerance.

It is clear that one tolerance mechanism might not be applied to a large range of compounds with highly variable physical and chemical properties. However, if the same cellular target is involved, e.g. cell-membrane, the same genes might still be involved in the tolerance mechanism although in different manners. In one embodiment of the invention the cellular target of the toxic compounds is the same, tolerance might be achieved by fine-tuning expression and function of genes disclosed in the present invention. This means that for membrane stress inducing compounds one membrane gene might need to be overexpressed or downregulated depending on the exact physical properties, but in each case the same target gene can be employed in one embodiment of the invention. In another embodiment the toolbox approach also presents targets for directed evolution approaches. Genes involved in a specific tolerance mechanism can be amplified using error-prone PCR approaches and selected on their benefit for tolerance.

TABLE 3B
Compounds with physical properties logP and solubility in water and the
tolerance of adapted strains or tested mutations.

| | | Solubility | | |
|---|---|---|---|---|
| Compound | loqP | g/L | Tolerance | Identified Beneficial Mutations |
| Isobutanol | 0.8 | 66.5 | ++ | |
| Butanol | 0.88 | 68 | ++ | yghB Expression, rob H48fs, |
| | | | | ΔmarC |
| Isoprenol | 0.89 | 90 | ++ | yghB Expression, rob H48fs, |
| | | | | ΔmarC, ArraA |
| Prenol | 0.91 | 170 | + | |
| Ethyl Propionate | 1.21 | 19 | | |
| 4-Hydroxybenzalde- | 1.35 | 8.45 | | |
| hyde | | | | |
| 2-Phenylethanol | 1.36 | 22 | | |
| Vanillin | 1.37 | 10 | + | ΔyghB, rob H48fs |
| Vanillic Acid | 1.43 | 1.5 | | |
| 4-(4-Hydroxyphenyl)- | 1.5 | 12.5 | | |
| 2-butanone | | | | |
| Acetophenone | 1.58 | 6.1 | | |
| Ethyl butanoate | 1.77 | 2.7 | | |
| Cinnamaldehyde | 1.9 | 1.1 | | |
| Geraniol | 2.5 | 0.68 | — | |
| (-)-Carvone | 2.71 | 1.3 | | |
| Citral | 2.9 | 0.42 | | |
| Linalool | 2.97 | 1.5 | | |
| Menthol | 3.2 | 0.45 | | |
| Farnesol | 4.16 | 0.05 | | |
| Limonene | 4.571 | 0.00757 | | |

The inventors have published some of these results (see Babel and Krömer 2020)

### 3 Experimental Materials & Methods

### 3.1 Strains, Plasmids and Primers

TABLE 4
Background strains

| Strain | Genotype | Reference |
|---|---|---|
| Escherichia coli MG | K-12 F⁻ λ⁻ ilvG⁻ rfb-50 rph-1 | — |
| 1655 | | |
| Escherichia coli DH5α | F− endA1 glnV44 thi-1 recA1 relA1 gyrA96 | — |
| | deoR nupG purB20 φ80dlacZΔM15 | |
| | Δ(lacZYA-argF)U169, hsdR17(rK−mK+), λ− | |
| Escherichia coli | rrnB3 DEIacZ4787 hsdR514 DE(araBAD)567 | (Baba et al., 2006) |
| BW25113 | DE(rhaBAD)568 rph-1 | |
| Keio ΔmarC | BW25113 marC::kan^{R} | (Baba et al., 2006) |
| Keio Δrob | BW25113 marC::rob^{R} | (Baba et al., 2006) |
| Keio ΔyghB | BW25113 marC::yghB^{R} | (Baba et al., 2006) |

TABLE 5
Strains constructed

| No. | strain |
|---|---|
| 1 | E. coli DH5α Ptrc10 yghB ampR cmR |
| 2 | E. coli MG1655 Ptrc10 yghB ampR cmR |
| 3 | E. coli BW25113 Ptrc10 empty ampR cmR |
| 4 | E. coli BW25113 Ptrc10 yghB ampR cmR |
| 5 | E. coli BW25113 ΔmarC750::kan Ptrc10 empty ampR cmR kanR |
| 6 | E. coli BW25113 ΔmarC750::kan Ptrc10 yghB ampR cmR kanR |
| 7 | E. coli BW25113 Δrob-721::kan Ptrc10 empty ampR cmR kanR |
| 8 | E. coli BW25113 Δrob-721::kan Ptrc10 yghB ampR cmR kanR |
| 9 | E. coli MG1655 Ptrc10 empty ampR cmR |
| 10 | E. coli MG1655 Ptrc10 robH ampR cmR |
| 11 | E. coli MG1655 ΔyghB781::kanR kanR |
| 12 | E. coli MG1655 ΔmarC750::kan kanR |
| 13 | E. coli MG1655 Δrob-721::kanR kanR |
| 14 | E. coli BW25113 ΔyghB781::kanR Ptrc10 yghB ampR cmR kanR |
| 15 | E. coli MG1655 Ptrc10 marC35 ampR cmR |
| 17 | E. coli BW25113 ΔyghB781::kanR Ptrc10 empty ampR cmR kanR |
| 18 | E. coli MG1655 ΔyghB781::FRT (Clean Deletion) |
| 19 | E. coli MG1655 Δrob-721::FRT (Clean Deletion) |
| 20 | E. coli MG1655 ΔmarC750::kanR Ptrc10 marC35 ampR cmR kanR |
| 21 | E. coli MG1655 Δrob-721::kanR Ptrc10 robH ampR cmR kanR |
| 22 | E. coli MG1655 ΔyghB781::kanR Ptrc10 yghB ampR cmR kanR |
| 23 | E. coli MG1655 ΔyghB781::kanR Ptrc10 empty ampR cmR kanR |
| 24 | E. coli MG1655 Δrob-721::kanR Ptrc10 empty ampR cmR kanR |
| 25 | E. coli MG1655 ΔmarC750::kanR Ptrc10 empty ampR cmR kanR |
| 26 | E. coli MG1655 Δrob-721::FRT (Clean Deletion) AmarC750::kan |
| 27 | E. coli MG1655 Δrob-721::FRT (Clean Deletion) AyghB781::kanR kanR |
| 28 | E. coli MG1655 Δrob-721::FRT (Clean Deletion) AmarC750::kan Ptrc10 robH ampR cmR kanR |

TABLE 6
Plasmids

| Plasmid | Genotype | Reference |
|---|---|---|
| pAH030 | P_{trc10} ori(pUC) cm^{R} amp^{R} laci | (Hoschek, Bühler |
| (SOMA81) | | and Schmid, 2017) |
| pHB01 | P_{trc10} yghB ori(pUC) cm^{R} amp^{R} laci | |
| pHB02 | P_{trc10} robH48-fs ori(pUC) cm^{R} amp^{R} | |
| | laci | |
| pHB03 | Pt_{trc10} marC ori(pUC) cm^{R} amp^{R} laci | |

TABLE 7
Primer

| Primer | | |
|---|---|---|
| No | Name | Sequence |
| 1 | yghB expr fwd | GGATAACAATTTCACACATACTAGTCGCTGTTCCACAGGAAAGTCC |
| 2 | yghB expr rev | CTTTCGTTTTATTTGATGCCTGGTACCTAGGCCGG-GAACGGGGAA |
| | | AATCG |
| 3 | rob del fwd | AATTACCTGATGTCAGGTGCTCGTTGTT-GAAAGGATGAGGATATT |
| | | TTATG |
| 4 | rob del rev | GACGCCCCTGCATTAGATGAGCTGCAGCGTTAAC-GACGGATCGGA |
| | | ATCAG |
| 5 | marC fwd | CTTATACTTTTCGCTGATAACCCAGATACACAGGA-TAACAACCAC |
| | | CAATG |
| 6 | marC rev | AATAGTT-GAAAGGCCCATTCGGGCCTTTTTTAATGGTAC-GTTTT |
| | | AATGAT |
| 7 | yghB del fwd | GTACAATAGGCAGATAAAGGCTTAAAC-GCTGTTCCACAGGAAAGT |
| | | CCATG |
| 8 | yghB del rev | CGGTACAGCAACCGGGAACGG-GAAAATCGTCAGGCGTTACAGATA |
| | | TTTTTT |
| 9 | fabF del fwd | TCTTTTTGTCCCACTA-GAATCATTTTTTCCCTCCCTGGAGGACAA |
| | | ACGTG |
| 10 | fabF del rev | GACCTTTTATAAGGGTGGAAATGACAACTTA-GATCTTTTTAAAGA |
| | | TCAA |
| 11 | plsX del fwd | TTTCCCCAGGCAACTGGGGAAAGACCAAAC-CGGGCGGCGACGATA |
| | | CCTTG |
| 12 | plsX del rev | CAAACTGCGAGTTCGCTGGCAGCGTCCTGCTAC-CGCAGAGTTCCG |
| | | CTTTT |
| 13 | rob H fwd | GGATAACAATTTCACACATACTAG-TCCTGATGTCAGGTGCTCGTT |
| 14 | rob H rev | CTTTCGTTTTATTTGATGCCTGGTACCTAGGTCAC-GAATACCAAA |
| | | GGCGCTCCA |
| 15 | marC35 fwd | GGATAACAATTTCACACATACTAGTTACACAGGA-TAACAACCACC |
| | | AATG |
| 16 | marC35 rev | CTTTCGTTTTATTTGATGCCTGGTACCTAGGTCAG-TTGCCTGCCA |
| | | GGCCAA |
| 17 | P yghB delta Homology | TTTATTGTGAAAAGTCTTAAATTGTCGTCCCGGAC-GATTCAGGAG |
| | fwd | TACAA |
| 18 | P yghB del Hom Amp-rev | CAGCGTGGCAAACATGACAA |
| 19 | P yghB del Hom Amp-fwd | TCTGATTGCCGATCTGGACG |
| 20 | P yghB del check-rev | CAGCAGCGTACGGACAAATG |
| 21 | P yghB del check-fwd | TCTTAAATTGTTGCGTCCCGG |
| 22 | P yghB CRISPR 20 nt | CTATTTCTAGCTCTAAAACGGATCAAGGCGTCCCGG |
| | rev | |
| 23 | P yghB CRISPR 20 nt | TAATACGACTCACTATAGCGTCCGGGACGCCTTG |
| | fwd | |
| 24 | Datsenko k1 | CAGTCATAGCCGAATAGCCT |
| 25 | Datsenko k2 | CGGTGCCCTGAATGAACTGC |
| 26 | rraA Keio 1 | ATAGCGCGATATACTGAAAATTCTCGCAG-CAACTGAATGTTAAGC |
| | | CTATG |
| 27 | rraA Keio 2 | AAAAAAGGCACCTT-GCGGTGCCTTTCTTATCATTCAATATCCAGC |
| | | GGATC |
| 28 | YghB Check Short Rev | GCGTTTAAGCCTTTATCTGCCT |
| 29 | YghB Check Short Fwd | ACTTTTGGACAATTTTGCAGACAT |
| 30 | Pyghb 50 bp Homology | TTATTGTGAAAAGTCTTAAATTGTTGCGTCCCGGAC-GATTCAGGA |
| | | GTACA |

These are included in the sequence listing as SEQ ID NO: 10 to 39

### 3.2 Strain and Plasmid Construction

### 3.2.1 Expression Plasmids

Knock-out strains were constructed by amplification of resistance cassette with 25 bp overlap from corresponding Keio strains (Primers 3+4, 5+6 and 7+8). The PCR products carrying a homologous 25 bp sequence and a kanamycin resistance were used to transform E. coli MG1655 using standard procedures (Baba et al., 2006). For over-expression plasmids target genes were amplified with a 25 bp homology to the pAH030 overexpression plasmid. The Plasmid was linearized using the Spel restriction site and the PCR-product containing the gene of interested was inserted using Gibson assembly (Gibson et al., 2009).

### 3.2.2 Knock-Out Strains

Knock out strains were prepared using DNA-fragments isolated from the corresponding Keio collection strains. Recombination was carried out using a standard RED/ET kit (Genebridges Red/ET Kit, 2019).

### 3.3 Cultivation of Microorganisms

### 3.3.1 Chemically Defined Media

For growth of E. coli M9 medium (Green and Sambrook, 2012) was used. M9 10× were adjusted to a pH of 7.

TABLE 8
M9 and M9* 10x concentrated solution

| | M9 Base |
|---|---|
| | 10x |
| Na₂HPO₄* | 85 g/L |
| H₂O | |
| KH₂PO₄ | 30 g/L |
| NaCl | 5 g/L |
| NH₄Cl | 10 g/L |

TABLE 9
Recipe for 1 L US Trace Metals (1000x)

| | |
|---|---|
| 37% fuming HCl | 82.81 mL |
| FeSO₄ * 7 H₂O | 4.87 g |
| CaCl₂ * 2 H2O | 4.12 g |
| MnCl₂ * 4 H2O | 1.50 g |
| ZnSO₄ * 7 H2O | 1.87 g |
| H₃BO₃ | 0.30 g |
| Na₂MoO₄ * 2 H₂O | 0.25 g |
| CuCl₂ * 2 H₂O | 0.15 g |
| Na₂EDTA * 2 H₂O | 0.84 g |

US trace metal solution was sterile filtered.

TABLE 10
Recipe for 1 L M9 or M9* Medium

| | |
|---|---|
| H₂O | 872 mL |
| M9 10x (or M9* 10x) | 100 mL |
| Glucose (20 % w/v) | 25 mL |
| US Trace Metals | 1 mL |
| 1 M MgSO₄ | 2 mL |

### 3.4 Cultivation Scheme and Conditions

### 3.4.1 Shake-Flask Based Evolution

E. coli was incubated at a temperature of 37° C.

Microorganisms are streaked out on a suitable chemically defined medium and grown at the optimal temperature. When colony formation was observed, 10 mL chemically defined medium were inoculated with a colony and incubated in a 100 mL baffled flask at a shaking speed of 200 rpm in an Infors HT Multitron (Switzerland, Bottmingen) or Ecotron (25 mm shaking throw). From this culture an over-night culture of 25 mL medium in a 250 mL baffled flask was inoculated and incubated for 16 hours, such that the culture is in mid-exponential phase at the next day.

On the next day 25 mL of medium in 250 mL baffled flasks with Teflon® liner screw cabs were inoculated to an OD of 0.2 and incubated at 200 rpm. The terpenoid stress was added to the specified concentration. Before the cell-culture reached stationary phase, part of the culture was transferred to fresh medium in a fresh flask with the terpenoid. The mean culture growth rate was determined by comparing the initial OD and the culture OD before passaging.

Before passaging the cell culture, 600 μL were withdrawn and mixed with 600 μL 50% v/v glycerol solution. The samples were stored at −80° C.

TABLE 11
Chemicals used

| Chemical | Vendor | Purity |
|---|---|---|
| Geraniol | MP Biomedicals | ≥98% |
| Isoprenol | Sigma | 97% |
| Isoprenol | BASF | Formaldehyde reduced |
| Isobutanol | Sigma | 99.5% |
| Butanol | Sigma | 99.7% |
| Vanillin | Sigma | 99% |

### 3.5 Evaluation of Growth Data

Growth rates were determined by transforming the OD-values of each experiment with the natural logarithm. In the linear regime of growth a line was fit to the data and the slope was determined, which is equal to the growth rate. Growth rates were determined for each flask individually and the growth rate of each condition is given as the mean and standard deviation of three biological replicates.

A linear interpolation of the two data-points adjacent to the half-maximal growth rate (MIC₅₀) was used to estimate the compound concentration of half-maximal growth rate. If the corresponding standard-deviations of the growth rates were available the standard error of the MIC₅₀ was computed by error propagation.

### 3.6 Propidium Iodine Staining

For propidium iodine staining cells were grown for 5 hours under the appropriate Isoprenol concentration in 250 mL baffled sealed shake-flasks. Of each condition 2 mL sample were taken and resuspended in the same volume of 0.85% NaCl solution. As a negative control wild-type sample were incubated with Bacillol AF for 5 minutes. After also washing the negative control, samples were diluted to a concentration of 1.5*10⁷ cells/mL and SYTO 9 50 μM stock-solution (in DMSO) and Propidium iodine 6 mM stock solution (in DMSO) were added to give a final concentration of 5 μM SYTO 9 and 6 μM Pl. SYTO 9 staining was used as a positive staining to distinguish cells from debris in the sample. The samples were incubated for at least 20 minutes at room temperature. Prior to measurement the samples were diluted to a final concentration of 1.5*10⁶ cells/mL. Samples were measured using a Beckman Coulter CytoFLEX Flow cytometer. Propidium iodine staining was detected exciting with a 488 nm laser and a 610/20 BP filter, SYTO 9 was measured using a 524/40 BP filter and the same excitation wavelength.

### 3.7 DNA and RNA Sequencing

### 3.7.1 DNA Sequencing

### 3.7.1.1 Sample Preparation

Selected strains were grown over night in 5 mL LB medium supplemented with 60 mM Isoprenol (for mutant strains). Genomic DNA was isolated using standard methods.

### 3.7.1.2 Library Preparation and Sequencing

1. DNA fragmentation using Covaris (desired fragment size: 300 bp)

2. Sample purification using using MinElute Columns (Qiagen), eluted in 20 μl EB buffer

3. Illumina library construction:
Preparing Indexed Illumina libraries using Ovation Rapid DR Multiplex System 1-96 (NuGEN) according to the manual

4. Library Amplification and size selection
Libraries were amplified for 13 cycles using MyTaq (Bioline) and standard Illumina primers. Size selection was done on the Pippin Prep system (Sage Science) selecting a range between 300 and 500 bp

5. Final library purification step and quality control of DNA libraries via BioAnalyzer and Qubit

6. Sequencing was done on an Illumina NextSeq 500/550-2×150 bp read length (Illumina)—followed manufactures instructions

### 3.7.1.3 Data Analysis

1. Read pre-processing:
Demultiplexing of all libraries for each sequencing lane using the Illumina bcl2fastq 2.17.1.14 software (folder ‘RAW’):
   1 or 2 mismatches or Ns were allowed in the barcode read when the barcode distances between all libraries on the lane allowed for it
Clipping of sequencing adapter remnants from all raw reads (folder ‘AdapterClipped’):
   reads with final length <20 bases were discarded
Quality trimming of adapter clipped Illumina reads (folder ‘QualityTrimmed’):
   removal of reads containing Ns
   trimming of reads at 3′-end to get a minimum average Phred quality score of 20 over a window of ten bases
   reads with final length<20 bases were discarded
Creation of FastQC reports for all FASTQ files
Generation of read_counts.xlsx, containing all read counts for all samples at a glance
2. Alignment and variant discovery:
Alignment of quality trimmed reads against the reference genome using BWA-MEM version 0.7.12 (http://bio-bwa.sourceforge.net/) (folder ‘Alignments’):
   one alignment file per sample in coordinate-sorted BAM format
Markup of PCR and optical duplicate reads with Picard v1.92 MarkDuplicates (http://picard.sourceforge.net/)
Variant discovery and genotyping of samples with Freebayes v1.0.2-16 (https://github.com/ekg/freebayes#readme) (folder VariantAnalysis/[reference]/Free-bayes’):
   reads with more than two mismatches were excluded
   MNPs and complex variants were excluded
   ploidy was set to 1

### 3.7.2 RNA Sequencing

### 3.7.2.1 Sample Preparation

Wild-type and the three final mutant strains were grown in biological triplicates with 50 mM Isoprenol until an OD of 1.0 as described above (25 mL sealed flasks). Then 10 mL of cell-culture was vacuum filtered using a Supor® 800 Grid filter with 0.8 μM pore size. The filter containing the cells was put in a 15 mL falcon tube containing 700 μL PGTX solution and immediately frozen in liquid nitrogen. The samples were stored at −80° C. until further processing.

To extract the RNA the samples were incubated for 15 min at 65° C. in a waterbath with occasional vortexing and then incubated for 5 min on ice. Then 700 μL of Chloroform was added and incubated for 10 min at room temperature. The samples were centrifuged for 15 minutes, the upper aqueous phase was transferred into a new vial and the same volume of chloroform was added. After mixing the sample was centrifuged for another 15 minutes. The upper aqueous phase (approx. 500 μL) was transferred to a new vial and mixed with the same volume of Isopropanol. The mixture was then incubated at −20° C. overnight.

On the next day the mixture was centrifuged for 30 minutes at 4° C. and 12000 g in a centrifuge that was cleaned with RNaseZAP. The supernatant was removed and the pellet was carefully washed (without resuspending the pellet) with 1 mL of 70% v/v Ethanol solution. After another centrifugation step for 5 minutes at 12000 g and 4° C. the pellet was dried with air for approximately 15 minutes under a clean bench. Finally the RNA was resuspended in RNAse-free water (40 μL). The sample concentration was determined using a Nanodrop. After treatment of the samples with Turbo DNA-free Kit (Invitrogen) the concentration was determined again and samples were inspected on a 1.5% Agarose native gel in TAE. The samples were stained using EZ-Vision Three staining.

### 3.7.2.2 Library Preparation and Sequencing

1. Quality control check of total RNA was performed via Bioanalyzer

2. rRNA depletion using Ribo-Zero rRNA Removal Kit for Bacteria (Illumina)—followed manufactures instructions

3. First strand cDNA synthesis—NEBNext RNA First Strand Synthesis Module (New England Biolabs) was used according to the manual

4. Second strand synthesis—NEBNext RNA Second Strand Synthesis Module (New England Biolabs) was used according to the manual

5. Purification and concentration of cDNA—cDNA from Step 5 was purified using MinElute Columns (Qiagen), eluted in 20 μl EB buffer

6. Illumina library construction
The Encore Rapid DR Multiplex system (Nugen) was used for library preparation according to the manual

7. Library Amplification and size selection
Libraries were amplified in a volume of 100 μl for 12 cycles using MyTaq (Bioline) and standard Illumina primers. Size selection was done on a preparative Agarose Gel, selecting fragments between 300 and 500 bp

8. Quality control of RNA libraries was performed via Bioanalyzer and Qubit

9. Sequencing on Illumina NextSeq500/550 (1×75 bp)—followed manufactures instructions

### 3.7.2.3 Data Analysis

1. Data pre-processing:
Demultiplexing of all libraries for each sequencing lane using the Illumina bcl2fastq 2.17.1.14 software (folder ‘RAW’):
   1 or 2 mismatches or Ns were allowed in the barcode read when the barcode distances between all libraries on the lane allowed for it
Clipping of sequencing adapter remnants from all raw reads (folder ‘AdapterClipped’):
   reads with final length<20 bases were discarded
Filtering of rRNA sequences using RiboPicker 0.4.3 (http://ribopicker.sourceforge.net/) (folder ‘RiboPicker’)
Generation of read_counts.xls, containing all read counts for all samples at a glance
Creation of FastQC reports for all FASTQ files
2. Differential expression analysis:
Alignment against reference with STAR 2.4. (https://github.com/alexdobin/STAR/releases) (folder ‘Alignments’)
Post-alignment filtering of reads aligning to rRNA or tRNA regions (folder ‘Alignments’)
Counting of TopHat-aligned reads with htseq-count (http://www-huber.embl.de/users/anders/HTSeq/) (folder ‘Alignments’)
Differential expression analysis with edgeR 3.2.3 (http://www.bioconductor.org/packages/release/bioc/html/edgeR.html), DESeq 1.12.0 (http://bioconductor.org/packages/release/bioc/html/DESeq.html) and cuffdiff 2.1.1 (http://cufflinks.cbcb.umd.edu) (folder ‘ExpressionAnalysis’, subfolders ‘edgeR’, ‘DESeq’ and ‘cuffdiff’):
   The raw p-values from the statistical tests were adjusted for multiple testing by the Benjamini-Hochberg false discovery rate (FDR) method

The inventors have published some of these results (see Babel and Krömer 2020)

### 4 REFERENCES

Aono, R. (1998) ‘Improvement of organic solvent tolerance level of Escherichia coli by overexpression of stress-responsive genes.’, Extremophiles: life under extreme conditions, 2(3), pp. 239-48. Available at: http://www.ncbi.nlm.nih.gov/pubmed/9783171 (Accessed: 27 Nov. 2018).
Baba, T. et al. (2006) ‘Construction of Escherichia coli K-12 in-frame, single-gene knockout mutants: the Keio collection.’, Molecular systems biology. EMBO Press, 2(1), p. 2006.0008. doi: 10.1038/msb4100050.
Babel, H and Krömer, J, (2020) ‘Evolutionary engineering of E. coli MG1655 for tolerance against isoprenol’, Biotechnol Biofuels (2020) 13:183; https://doi.org/10.1186/s13068-020-01825-6
Brennan, T. C. R. et al. (2012) ‘Alleviating monoterpene toxicity using a two-phase extractive fermentation for the bioproduction of jet fuel mixtures in Saccharomyces cerevisiae’, Biotechnology and Bioengineering. Wiley Subscription Services, Inc., A Wiley Company, 109(10), pp. 2513-2522. doi: 10.1002/bit.24536.
Brennan, T. C. R. et al. (2015) ‘Evolutionary Engineering Improves Tolerance for Replacement Jet Fuels in Saccharomyces cerevisiae.’, Applied and environmental microbiology. American Society for Microbiology, 81(10), pp. 3316-25. doi: 10.1 128/AEM.04144-14.
Brosius, J., Erfle, M. and Storella, J. (1985) ‘Spacing of the −10 and −35 regions in the tac promoter. Effect on its in vivo activity.’, The Journal of biological chemistry, 260(6), pp. 3539-41. Available at: http://www.ncbi.nlm.nih.gov/pubmed/2579077 (Accessed: 12 Feb. 2019). Cao, Y. et al. (2011) ‘Crystal structure of a potassium ion transporter, TrkH.’, Nature. NIH Public Access, 471(7338), pp. 336-40. doi: 10.1038/nature09731.
Duckworth, H. W. et al. (2013) ‘Enzyme-substrate complexes of allosteric citrate synthase: Evidence for a novel intermediate in substrate binding’, Biochimica et Biophysica Acta—Proteins and Proteomics. Elsevier B. V., 1834(12), pp. 2546-2553. doi: 10.1016/j.bbapap.2013.07.019. Freddolino, P. L., Amini, S. and Tavazoie, S. (2012) ‘Newly identified genetic variations in common Escherichia coli MG1655 stock cultures.’, Journal of bacteriology. American Society for Microbiology Journals, 194(2), pp. 303-6. doi: 10.1128/JB.06087-11.
Genebridges Red/ET Kit (2019). Available at: https://www.genebridges.com/products/redet-kits. George, K. W. et al. (2018) ‘Integrated analysis of isopentenyl pyrophosphate (IPP) toxicity in isoprenoid-producing Escherichia coli’, Metabolic Engineering. Elsevier Inc. doi: 10.1016/j.ymben.2018.03.004.
Gibson, D. G. et al. (2009) ‘Enzymatic assembly of DNA molecules up to several hundred kilobases’, Nature Methods. Nature Publishing Group, 6(5), pp. 343-345. doi: 10.1038/nmeth.1318.
Green, M. R. and Sambrook, J. (2012) Molecular Cloning: A Laboratory Manual (Fourth Edition), Cold Spring Harbor Laboratory Press; 4th edition (Jun. 15, 2012).
Griffith, K. L. et al. (2009) ‘Two Functions of the C-Terminal Domain of Escherichia coli Rob: Mediating “Sequestration-Dispersal” as a Novel Off-On Switch for Regulating Rob's Activity as a Transcription Activator and Preventing Degradation of Rob by Lon Protease’, Journal of Molecular Biology. Elsevier Ltd, 388(3), pp. 415-430. doi: 10.1016/j.jmb.2009.03.023.
Gupta, S. et al. (2007) ‘Quantifying similarity between motifs’, Genome Biology. BioMed Central, 8(2), p. R24. doi: 10.1186/gb-2007-8-2-r24.
Haeyoung, J. and Jihee, H. (2010) ‘Enhancing 1-Butanol Tolerance in Escherichia coli through Repetitive Proton Beam Irradiation’, Journal of the Korean Physical Society, 56(61), p. 2041. doi: 10.3938/jkps.56.2041.
Heipieper, H. J. et al. (1994) ‘Mechanisms of resistance of whole cells to toxic organic solvents’, Trends in Biotechnology, 12(10), pp. 409-415. Available at: https://ac.elscdn. com/0167779994900299/1-s2.0-0167779994900299-main.pdf?_tid=efcbc9f6-4230-4f02-aa88-78d5d226a6c0&acdnat=1543229509_Of363d9a2174923613184795e30d8ea7 (Accessed: 26 Nov. 2018).
van Helden, J., Andre, B. and Collado-Vides, J. (1998) ‘Extracting regulatory sites from the upstream region of yeast genes by computational analysis of oligonucleotide frequencies 1 1Edited by G. von Heijne’, Journal of Molecular Biology, 281(5), pp. 827-842. doi: 10.1006/jmbi.1998.1947.
Hoschek, A., BQhler, B. and Schmid, A. (2017) ‘Overcoming the Gas-Liquid Mass Transfer of Oxygen by Coupling Photosynthetic Water Oxidation with Biocatalytic Oxyfunctionalization’, Angewandte Chemie International Edition. John Wiley & Sons, Ltd, 56(47), pp. 15146-15149. doi: 10.1002/anie.201706886.
Janssen, T. (2015)/sobionics Presentation Food Valley Expo 15.
Jeong, H. et al. (2012) ‘Changes in membrane fatty acid composition through proton-induced fabF mutation enhancing 1-butanol tolerance in E. coli’, Journal of the Korean Physical Society, 61(2), pp. 227-233. doi: 10.3938/jkps.61.227.
Kang, A. et al. (2017) ‘High-throughput enzyme screening platform for the IPP-bypass mevalonate pathway for isopentenol production’, Metabolic Engineering. Elsevier Inc., 41, pp. 125-134. doi: 10.1016/j.ymben.2017.03.010.
Kang, A et al. (2019) “Optimization of the IPP-bypass mevalonate pathway and fed-batch fermentation for the production of isoprenol in Escherichia coli”, Metabolic Engineering, Volume 56, pp 85-96, https://doi.org/10.1016/j.ymben.2019.09.003.
Kumar, S. and Doerrler, W. T. (2014) ‘Members of the conserved DedA family are likely membrane transporters and are required for drug resistance in Escherichia coli.’, Antimicrobial agents and chemotherapy. American Society for Microbiology (ASM), 58(2), pp. 923-30. doi: 10.1 128/AAC.02238-13.
Lee, K. et al. (2003) ‘RraA: a Protein Inhibitor of RNase E Activity that Globally Modulates RNA Abundance in E. coli’, Cell. Cell Press, 114(5), pp. 623-634. doi: 10.1016/J.CELL.2003.08.003.
Liu, H. et al. (2014) ‘MEP pathway-mediated isopentenol production in metabolically engineered Escherichia coli’, Microbial Cell Factories. BioMed Central, 13(1), p. 135. doi: 10.1186/s12934-014-0135-y.
Minty, J. J. et al. (2011) ‘Evolution combined with genomic study elucidates genetic bases of isobutanol tolerance in Escherichia coli’, Microbial Cell Factories. BioMed Central, 10(1), p. 18. doi: 10.1186/1475-2859-10-18.
Monzingo, A. F. et al. (2003) ‘The X-ray structure of Escherichia coli RraA (MenG), a protein inhibitor of RNA processing’, Journal of Molecular Biology. Academic Press, 332(5), pp. 1015-1024. doi: 10.1016/S0022-2836(03)00970-7.
Pandey, S. et al. (2019) ‘3-Methyl-3-buten-1-ol (isoprenol) confers longevity and stress tolerance in Caenorhabditis elegans’, International Journal of Food Sciences and Nutrition. Taylor & Francis, pp. 1-8. doi: 10.1080/09637486.2018.1554031.
Rosenberg, E. Y. et al. (2003) ‘Bile salts and fatty acids induce the expression of Escherichia coli AcrAB multidrug efflux pump through their interaction with Rob regulatory protein’, Molecular Microbiology. Wiley/Blackwell (10.1111), 48(6), pp. 1609-1619. doi: 10.1046/j.1365-2958.2003.03531.x.
Wang, S. et al. (2019) ‘NaCl enhances Escherichia coli growth and isoprenol production in the presence of imidazolium-based ionic liquids’, Bioresource Technology Reports. Elsevier, 6, pp. 1-5. doi: 10.1016/J.BITEB.2019.01.021.
White, D. G. et al. (1997) ‘Role of the acrAB locus in organic solvent tolerance mediated by expression of marA, soxS, or robA in Escherichia coli.’, Journal of bacteriology. American Society for Microbiology Journals, 179(19), pp. 6122-6. doi: 10.1128/JB.179.19.6122-6126.1997.
Withers, S. T. and Keasling, J. D. (2006) ‘Biosynthesis and engineering of isoprenoid small molecules’, Applied Microbiology and Biotechnology. Springer-Verlag, 73(5), pp. 980-990. doi: 10.1007/s00253-006-0593-1.
Yao, J. and Rock, C. O. (2013) ‘Phosphatidic acid synthesis in bacteria.’, Biochimica et biophysica acta. NIH Public Access, 1831(3), pp. 495-502. doi: 10.1016/j.bbalip.2012.08.018.

### Further Aspects of the Invention

Preferably the growth rate in the presence of the toxic substances like terpenes is increased by 5%, 10% or 15%, more preferably by 20%, 25%, 30%, 35%, 40%, 45% or 50 or more % compared to the control, i.e. the unmodified organisms.

More preferably, the growth rate in the presence of the toxic substances like terpenes is improved by a factor of 1.1, 1.2, 1.25, 1.3, 1.4, 1.5, 1.75, 2, 3, 4, 5, 6, 7, 8, 9, or 10.

Particularly useful in the methods and modified organisms of the invention are modifications corresponding to the modifications of Escherichia coli of the present invention, preferably that correspond to the disclosed modifications in those genes that encode proteins as provided in SEQ ID NOs:1 to 9 or of at least 60%, 65%, 70%, 75%, 80%, 85%, 90%, 95% or 98% sequence identity to these.

Unless otherwise noted, the terms used herein are to be understood according to conventional usage by those of ordinary skill in the relevant art. In addition to the definitions of terms provided herein, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement).

It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limit-ing.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in M. Green & J. Sambrook (2012) Molecular Cloning: a laboratory manual, 4th Edition Cold Spring Harbor Laboratory Press, CSH, New York; Ausubel et al., Current Protocols in Molecular Biology, Wiley Online Library; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York.

If not stated otherwise herein, abbreviations and nomenclature, where employed, are deemed standard in the field and commonly used in professional journals such as those cited herein.

Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are known in the art (see, e.g., Current Protocols in Molecular Biology (F. M. Ausubel et al. (eds) Chapter 9, 1987; Sambrook et al, Molecular Cloning: A Laboratory Manual, 2nd ed., Cold Spring Harbor, 1989; and Campbell et al, Curr. Genet. 16:53-56, 1989, which are each hereby incorpo-rated by reference in their entireties, particularly with respect to transformation methods). The expression of heterologous polypeptide in Trichoderma is described in U.S. Pat. Nos. 6,022,725; 6,268,328; 7,262,041; WO 2005/001036; Harkki et al., Enzyme Microb. Technol. 13:227-233, 1991; Harkki et al, Bio Technol 7:596-603, 1989; EP 244,234; EP 215,594; and Nevalainen et al, “The Molecular Biology of Trichoderma and its application to the Expression of Both Homologous and Heterologous Genes,” in Molecular Industri-al Mycology, Eds. Leong and Berka, Marcel Dekker Inc., NY pp. 129-148, 1992, which are each hereby incorporated by reference in their entireties, particularly with respect to transfor-mation and expression methods). Reference is also made to Cao et al, (Sd. 9:991-1001, 2000; EP 238023; and Yelton et al, Proceedings. Natl. Acad. Sci. USA 81:1470-1474, 1984 (which are each hereby incorporated by reference in their entireties, particularly with respect to transformation methods) for transformation of Aspergillus strains. The introduced nucleic acids may be integrated into chromosomal DNA or maintained as extrachromosomal replicating sequences.

In one embodiment, the invention relates to isolated genes and/or isolated proteins encoded by these that convey increased tolerance to terpene compounds, preferably monoterpene compounds, to an organism or a host cell. Included are variants of the genes and proteins as well as variants thereof and nucleic acid hybridising to the nucleic acids of such ability described herein, wherein these variants and hybridising sequences of the invention convey a protective effect towards terpene compounds to an organism or a host cell that is at least substantially as high as the protective effect of the nucleic acids of the invention.

The term “gene” means the segment of DNA involved in producing a polypeptide chain; it includes regions preceding and following the coding region (leader and trailer) as well as intervening sequences (introns) between individual coding segments (exons). Typically this is a segment of DNA containing hereditary information that is passed on from parent to offspring and that contributes to the phenotype of an organism. The influence of a gene on the form and function of an organism is mediated through the transcription into RNA (tRNA, rRNA, mRNA, non-coding RNA) and in the case of mRNA through translation into pep-tides and proteins.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are se-lected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984):

Tm=81.5° C.+16.6xlog[Na+]a+0.41x %[G/Cb]−500x[Lc]−1−0.61x % formamide
DNA-RNA or RNA-RNA hybrids:

Tm=79.8+18.5(log 10[Na+]a)+0.58(% G/Cb)+11.8(% G/Cb)2−820/Lc
oligo-DNA or oligo-RNAd hybrids:

For <20 nucleotides: Tm=2 (In)

For 20-35 nucleotides: Tm=22+1.46 (In)

a or for other monovalent cation, but only accurate in the 0.01-0.4 M range.
b only accurate for % GC in the 30% to 75% range.
c L=length of duplex in base pairs.
d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybrid-isation, samples are washed with dilute salt solutions. Critical factors of such washes include the ionic strength and temperature of the final wash solution: the lower the salt concentration and the higher the wash temperature, the higher the stringency of the wash. Wash conditions are typical-ly performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Recombinant” (or transgenic) with regard to a cell or an organism means that the cell or organism contains an exogenous polynucleotide which is introduced by gene technology and with regard to a polynucleotide means all those constructions brought about by gene technology/recombinant DNA techniques in which either

(a) the sequence of the polynucleotide or a part thereof, or
(b) one or more genetic control sequences which are operably linked with the polynucleotide, for example a promoter, or
(c) both a) and b)
are not located in their wildtype genetic environment or have been modified.

It shall further be noted that the term “isolated nucleic acid” or “isolated polypeptide” may in some instances be considered as a synonym for a “recombinant nucleic acid” or a “recombinant polypeptide”, respectively and refers to a nucleic acid or polypeptide that is not located in its natural genetic environment or cellular environment, respectively, and/or that has been modified by recombinant methods. An isolated nucleic acid sequence or isolated nucleic acid molecule is one that is not in its native surrounding or its native nucleic acid neighborhood, yet it is physically and functionally connected to other nucleic acid sequences or nucleic acid molecules and is found as part of a nucleic acid construct, vector sequence or chromosome. Typically, the isolated nucleic acid is obtained by isolating RNA from cells under laboratory conditions and converting it in copy-DNA (cDNA).

“Parent” (or “reference” or “template”) of a nucleic acid, protein, enzyme, or organism (also called “parent nucleic acid”, “reference nucleic acid”, “template nucleic acid”, “parent protein” “reference protein”, “template protein”, “parent enzyme” “reference enzyme”, “template enzyme”, “parent organism” “reference organism”, or “template organism”)) is the starting point for the introduction of changes (e.g. by introducing one or more nucleic acid or amino acid substitutions) resulting in “variants” of the parent. Thus, terms such as “enzyme variant” or “sequence variant” or “variant protein” are used to distinguish the modified or variant sequences, proteins, enzymes, or organisms from the parent sequences, proteins, enzymes, or organisms that are the origin for the respective variant sequences, proteins, enzymes, or organisms. There-fore, parent sequences, proteins, enzymes, or organisms include wild type sequences, proteins, enzymes, or organisms, and variants of wild-type sequences, proteins, enzymes, or organisms which are used for development of further variants. Variant proteins or enzymes differ from parent proteins or enzymes in their amino acid sequence to a certain extent; however, variants at least maintain the functional properties, e.g., enzyme properties, of the respective parent. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

In describing the variants, the nomenclature described as follows is used: Abbreviations for single amino acids used within this invention are according to the accepted IUPAC single letter or three letter amino acid abbreviation. While the definitions below describe variants in the context of amino acid changes, nucleic acids may be similarly modified, e.g. by substitutions, deletions, and/or insertions of nucleotides.

“Substitutions” are described by providing the original amino acid followed by the number of the position within the amino acid sequence, followed by the substituted amino acid. For example, the substitution of histidine at position 120 with alanine is designated as “Hisl20Ala” or “H120A”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G180GKA.

In cases where a substitution and an insertion occur at the same position, this may be indicated as S99SD+S99A or in short S99AD.

In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g. “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma e.g. R170Y G195E or R170Y, G195E respectively.

Where different alterations can be introduced at a position, the different alterations are separated by a comma, e.g. “Arg170Tyr, Glu” represents a substitution of arginine at position 170 with tyro-sine or glutamic acid. Alternatively, different alterations or optional substitutions may be indicated in brackets e.g. Arg170[Tyr, Gly] or Arg170{Tyr, Gly} or in short R170 [Y,G] or R170 {Y, G}.

Variants may include one or more alterations, either of the same type, e.g., all substitutions, or combinations of substitutions, deletions, and/or insertions. Alterations can be introduced to the nucleic acid or to the amino acid sequence.

In one embodiment, the sequence variant (i.e. amino acid sequence variant or nucleic acid sequence variant) includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, or more alterations.

Variants include nucleic acids and polypeptides having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to any of SEQ ID NO: 1 to 9 10 or 10 to 1820, respectively.

For substituting amino acids of a base sequence selected from any of the sequences SEQ ID NO. 1 to 9 without regard to the occurrence of amino acids in other of these sequences, the following applies, wherein letters indicate L amino acids using their common abbreviation and bracketed numbers indicate preference of replacement (higher numbers indicate higher preference): A may be replaced by any amino acid selected from S (1), C (0), G (0), T (0) or V (0). C may be replaced by A (0). D may be replaced by any amino acid selected from E (2), N (1), Q (0) or S (0). E may be replaced by any amino acid selected from D (2), Q (2), K (1), H (0), N (0), R (0) or S (0). F may be replaced by any amino acid selected from Y (3), W (1), I (0), L (0) or M (0). G may be replaced by any amino acid selected from A (0), N (0) or S (0). H may be replaced by any amino acid selected from Y (2), N (1), E (0), Q (0) or R (0). I may be replaced by any amino acid selected from V (3), L (2), M (1) or F (0). K may be replaced by any amino acid selected from R (2), E (1), Q (1), N (0) or S (0). L may be replaced by any amino acid selected from I (2), M (2), V (1) or F (0). M may be replaced by any amino acid selected from L (2), 1 (1), V (1), F (0) or Q (0). N may be replaced by any amino acid selected from D (1), H (1), S (1), E (0), G (0), K (0), Q (0), R (0) or T (0). Q may be replaced by any amino acid selected from E (2), K (1), R (1), D (0), H (0), M (0), N (0) or S (0). R may be replaced by any amino acid selected from K (2), Q (1), E (0), H (0) or N (0). S may be replaced by any amino acid selected from A (1), N (1), T (1), D (0), E (0), G (0), K (0) or Q (0). T may be replaced by any amino acid selected from S (1), A (0), N (0) or V (0). V may be replaced by any amino acid selected from I (3), L (1), M (1), A (0) or T (0). W may be replaced by any amino acid selected from Y (2) or F (1). Y may be replaced by any amino acid selected from F (3), H (2) or W (2).

Nucleic acids and polypeptides may be modified to include tags or domains. Tags may be utilized for a variety of purposes, including for detection, purification, solubilization, or immobilization, and may include, for example, biotin, a fluorophore, an epitope, a mating factor, or a regula-tory sequence. Domains may be of any size and which provides a desired function (e.g., imparts increased stability, solubility, activity, simplifies purification) and may include, for example, a binding domain, a signal sequence, a promoter sequence, a regulatory sequence, an N-terminal extension, or a C30 terminal extension. Combinations of tags and/or domains may also be utilized.

“Enzymatic activity” means at least one catalytic effect exerted by an enzyme. In one embodiment, enzymatic activity is expressed as units per milligram of enzyme (specific activity) or molecules of substrate transformed per minute per molecule of enzyme (molecular activity.

Alignment of sequences is preferably done with the algorithm of Needleman and Wunsch Needleman and Wunsch algorithm—Needleman, Saul B. & Wunsch, Christian D. (1970). “A general method applicable to the search for similarities in the amino acid sequence of two proteins”. Journal of Molecular Biology. 48 (3): 443-453. This algorithm is, for example, implemented into the “NEEDLE” program, which performs a global alignment of two sequences. The NEEDLE program, is contained within, for example, the European Molecular Biology Open Software Suite (EMBOSS), a collection of various programs: The European Molecular Biology Open Software Suite (EMBOSS), Trends in Genetics 16 (6), 276 (2000).

A number of techniques for targeted modification in a genome of an organism are known. Most widely known is the technology known as CRIPR or CRISPR/CAS:

The CRISPR (clustered regularly interspaced short palindromic repeats) technology may be used to modify the genome of a target organism, for example to introduce any given DNA fragment into nearly any site of the genome, to replace parts of the genome with desired sequences or to precisely delete a given region in the genome of a target organism. This allows for unprecedented precision of genome manipulation.

The CRISPR system was initially identified as an adaptive defense mechanisms of bacteria belonging to the genus of Streptococcus (WO2007/025097). Those bacterial CRISPR systems rely on guide RNA (gRNA) in complex with cleaving proteins to direct degradation of complementary sequences present within invading viral DNA. The application of CRISPR systems for genetic manipulation in various eukaryotic organisms have been shown (WO2013/141680; WO2013/176772; WO2014/093595). Cas9, the first identified protein of the CRISPR/Cas system, is a large monomeric DNA nuclease guided to a DNA target sequence adjacent to the PAM (protospacer adjacent motif) sequence motif by a complex of two noncoding RNAs: CRSIPR RNA (crRNA) and trans-activating crRNA (tracrRNA). Also a synthetic RNA chimera (single guide RNA or sgRNA) created by fusing crRNA with tracrRNA was shown to be equally func-tional (WO2013/176772). CRISPR systems from other sources comprising DNA nucleases dis-tinct from Cas9 such as Cpf1, C2c1p or C2c3p have been described having the same function-ality (WO2016/0205711, WO2016/205749). Other authors describe systems in which the nucle-ase is guided by a DNA molecule instead of an RNA molecule. Such system is for example the AGO system as disclosed in US2016/0046963.

Several research groups have found that the CRISPR cutting properties could be used to disrupt target regions in almost any organism's genome with unprecedented ease. Recently it became clear that providing a template for repair allows for editing the genome with nearly any desired sequence at nearly any site, transforming CRISPR into a powerful gene editing tool (WO2014/150624, WO2014/204728). The template for repair is addressed as donor nucleic acid comprising at the 3′ and 5′ end sequences complementary to the target region allowing for ho-mologous recombination in the respective template after introduction of doublestrand breaks in the target nucleic acid by the respective nuclease.

The main limitation in choosing the target region in a given genome is the necessity of the presence of a PAM sequence motif close to the region where the CRISPR related nuclease introduces doublestrand breaks. However, various CRISPR systems recognize different PAM sequence motifs. This allows choosing the most suitable CRISPR system for a respective target region. Moreover, the AGO system does not require a PAM sequence motif at all.

The technology may for example be applied for alteration of gene expression in any organism, for example by exchanging the promoter upstream of a target gene with a promoter of different strength or specificity. Other methods disclosed in the prior art describe the fusion of activating or repressing transcription factors to a nuclease minus CRISPR nuclease protein. Such fusion proteins may be expressed in a target organism together with one or more guide nucleic acids guiding the transcription factor moiety of the fusion protein to any desired promoter in the target organism (WO2014/099744; WO2014/099750). Knockouts of genes may easily be achieved by introducing point mutations or deletions into the respective target gene, for example by inducing non-homologous-end-joining (NHEJ) which usually leads to gene disruption (WO2013/176772).

“Modified organism” is an organism that has been modified, isolated, selected and/or domesticated by human intervention and differs from the organism as it occurred or occurs in the wild. Modified organisms include recombinant organisms and host cells as defined herein, but also mutated organisms without the use of gene editing or without the recombinant elements anymore for example without the CRISPR technology used to generate a mutated organism.

### “Host Cells”

Host cells also called host organisms may be any cell selected from bacterial cells, yeast cells, fungal, algal or cyanobacterial cells, non-human animal or mammalian cells, or plant cells. The skilled artisan is well aware of the genetic elements that must be present on the genetic construct to successfully transform, select and propagate host cells containing the sequence of interest.

In one embodiment host cell or host organisms are used interchangeably.

Typical host cells or modified organisms are Bacteria, such as gram positive: Bacillus, Streptomyces. Useful gram positive bacteria include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus iautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus. Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Further typical host cells or modified organisms are gram negative: E. coli, Pseudomonas, preferred gram negative bacteria are Escherichia coli and Pseudomonas sp., preferably, Pseudomonas purrocinia (ATCC 15958) or Pseudomonas fluorescens (NRRL B-11).

Further typical host cells or modified organisms are fungi, such as Aspergillus, Fusarium, Trichoderma. The microor-ganism may be a fungal cell. “Fungi” as used herein includes the phyla Ascomycota, Basidiomy-cota, Chytridiomycota, and Zygomycota as weil as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed be-low. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).

Further preferred fungi include strains belonging to the subdivision Zygomycotina, class My-coraceae, e.g. Rhizopus or Mucor, in particular Mucor hiemalis.

Further typical host cells or modified organisms are yeasts. Such as Pichia species or Saccharomyces species. The fungal host cell may be a yeast cell. “Yeast” as used herein includes ascosporogenous yeast (Endomycetales), basidiosporogenous yeast, and yeast belonging to the Fungi Imperfecti (Blas-tomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts in-clude the genera Leucosporidim, Rhodosporidium, Sporidiobolus, Filobasidium, and Filobasidiel-la. Yeasts belonging to the Fungi Imperfecti are divided into two families, Sporobolomycetaceae (e.g., genera Sporobolomyces and Bullera) and Cryptococcaceae (e.g. genus Candida).

Also typical host cells or modified organisms are Eukaryotes such as non-human animal, nonhuman mammal, avian, reptilian, insect, plant, yeast, fungi or plants.

In one embodiment the modified organism is a prokaryotic microorganism.

Preferably the host organism or modified organism according to the invention can be a gram positive or gram negative prokaryotic microorganism.

Useful gram positive prokaryotic microorganism include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus iautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacil-lus subtilis, Bacillus pumilus, Bacillus licheniformis, or Bacillus lentus. Some other preferred bacteria include strains of the order Actinomycetales, preferably, Streptomyces, preferably Strep-tomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Further typical prokaryotic organisms are gram negative: Escherichia. coli, Pseudomonas, preferred gram negative prokaryotic microorganisms are Escherichia coli and Pseudomonas sp., preferably, Pseudomonas purrocinia (ATCC 15958) or Pseudomonas fluorescens (NRRL B-11).

Most preferably the prokaryotic microorganism is Escherichia coli.

The terms “increase”, “improve” or “enhance” in the context of decreasing sensitivity to and increasing growth in the presence of toxic substances like terpenes are interchangeable and shall mean in the sense of the application at least a 3%, 4%, 5%, 6%, 7%, 8%, 9% or 10%, preferably at least 15% or 20%, more preferably 25%, 30%, 35% or 40% increase in comparison to controls as defined herein.

Culturing a microorganism frequently requires that cells be cultured in a medium containing various nutrition sources, like a carbon source, nitrogen source, and other nutrients, including but not limited to amino acids, vitamins, minerals, required for growth of those cells. The fermentation medium may be a minimal medium as described in, e.g., WO 98/37179, or the fermentation medium may be a complex medium comprising complex nitrogen and carbon sources, wherein the complex nitrogen source may be partially hydrolysed as described in WO 2004/003216.

Thus, fermentation medium comprises components required for the growth of the cultivated microorganism. In one embodiment, the fermentation medium comprises one or more components selected from the group consisting of nitrogen source, phosphor source, sulphur source and salt, and optionally one or more further components selected the group consisting of micronutrients, like vitamins, amino acids, minerals, and trace elements. In one embodiment, the fermentation medium also comprises a carbon source. Such components are generally well known in the art (see, e.g., Ausubel, et al, Short Protocols in Molecular Biology, 3rd ed., Wiley & Sons, 1995; Sambrook, et al., Molecular Cloning: A Laboratory Manual, Second Edition, 1989 Cold Spring Harbor, N.Y.; Talbot, Molecular and Cellular Biology of Filamentous Fungi: A Practical Ap-proach, Oxford University Press, 2001; Kinghom and Turner, Applied Molecular Genetics of Filamentous Fungi, Cambridge University Press, 1992; and Bacillus (Biotechnology Handbooks) by Colin R. Harwood, Plenum Press, 1989). Culture conditions for a given cell type may also be found in the scientific literature and/or from the source of the cell such as the American Type Culture Collection (ATCC) and Fungal Genetics Stock Center.

As sources of nitrogen, inorganic and organic nitrogen compounds may be used, both individual-ly and in combination. Suitable organic nitrogen sources include but are not limited to protein-containing substances, such as an extract from microbial, animal or plant cells, including but not limited thereto plant protein preparations, soy meal, corn meal, pea meal, corn gluten, cotton meal, peanut meal, potato meal, meat and casein, gelatines, whey, fish meal, yeast protein, yeast extract, tryptone, peptone, bacto-tryptone, bacto-peptone, wastes from the processing of microbial cells, plants, meat or animal bodies, and combinations thereof. Inorganic nitrogen sources include but are not limited to ammonium, nitrate, and nitrite, and combinations thereof. In one embodiment, the fermentation medium comprises a nitrogen source, wherein the nitrogen source is a complex or a defined nitrogen source or a combination thereof. In one embodiment, the com-plex nitrogen source is selected from the group consisting of plant protein, including but not limited to, potato protein, soy protein, corn protein, peanut, cotton protein, and/or pea protein, ca-sein, tryptone, peptone and yeast extract and combinations thereof. In one embodiment, the de-fined nitrogen source is selected from the group consisting of ammonia, ammonium, ammonium salts, (e.g., ammonium chloride, ammonium nitrate, ammonium phosphate, ammonium sulphate, ammonium acetate), urea, nitrate, nitrate salts, nitrite, and amino acids, including but not limited to glutamate, and combinations thereof.

In one embodiment, the fermentation medium further comprises at least one carbon source. The carbon source can be a complex or a defined carbon source or a combination thereof. Various sugars and sugar-containing substances are suitable sources of carbon, and the sugars may be present in different stages of polymerisation. The complex carbon sources include, but are not limited thereto, molasse, corn steep liquor, cane sugar, dextrin, starch, starch hydrolysate, and cellulose hydrolysate, and combinations thereof. The defined carbon sources include, but are not limited thereto, carbohydrates, organic acids, and alcohols. In one embodiment, the defined car-bon sources include, but are not limited thereto, glucose, fructose, galactose, xylose, arabinose, sucrose, maltose, lactose, gluconate, acetic acid, propionic acid, lactic acid, formic acid, malic acid, citric acid, fumaric acid, glycerol, inositol, mannitol and sorbitol, and combinations thereof. In one embodiment, the defined carbon source is provided in form of a syrup, which can com-prise up to 20%, up to 10%, or up to 5% impurities. In one embodiment, the carbon source is sugar beet syrup, sugar cane syrup, corn syrup, including but not limited to, high fructose corn syrup. The complex carbon source includes, but is not limited to, molasses, corn steep liquor, dextrin, and starch, or combinations thereof. In a preferred embodiment the defined carbon source includes, but is not limited to, glucose, fructose, galactose, xylose, arabinose, sucrose, maltose, dextrin, lactose, gluconate or combinations thereof.

In one embodiment, the fermentation medium also comprises a phosphor source, including, but not limited to, phosphate salts, and/or a sulphur source, including, but not limited to, sulphate salts. In one embodiment, the fermentation medium also comprises a salt. In one embodiment, the fermentation medium comprises one or more inorganic salts, including, but not limited to al kali metal salts, alkali earth metal salts, phosphate salts and sulphate salts. In one embodiment, the one or more salt includes, but is not limited to, NaCl, KH2PO4, MgSO4, CaCl2, FeC13, MgCl2, MnC12, ZnSO4, Na2MoO4 and CuSO4. In one embodiment, the fermentation medium also comprises one or more vitamins, including, but not limited to, thiamine chloride, biotin, vitamin B12. In one embodiment, the fermentation medium also comprises trace elements, including, but not limited to, Fe, Mg, Mn, Co, and Ni. In one embodiment, the fermentation medium comprises one or more salt cations selected from the group consisting of Na, K, Ca, Mg, Mn, Fe, Co, Cu, and Ni. In one embodiment, the fermentation medium comprises one or more divalent or trivalent cations, including but not limited to, Ca and Mg.

In one embodiment, the fermentation medium also comprises an antifoam.

In one embodiment, the fermentation medium also comprises a selection agent, including, but not limited to, an antibiotic, including, but not limited to, ampicillin, tetracycline, kanamycin, hygromycin, bleomycin, chloroamphenicol, streptomycin or phleomycin or a herbicide, to which the selectable marker of the cells provides resistance.

The fermentation may be performed as a batch, a repeated batch, a fed-batch, a repeated fedbatch or a continuous fermentation process. In a fed-batch process, either none or part of the compounds comprising one or more of the structural and/or catalytic elements, like carbon or nitrogen source, is added to the medium before the start of the fermentation and either all or the remaining part, respectively, of the compounds comprising one or more of the structural and/or catalytic elements are fed during the fermentation process. The compounds which are selected for feeding can be fed together or separate from each other to the fermentation process. In a repeated fed-batch or a continuous fermentation process, the complete start medium is additionally fed during fermentation. The start medium can be fed together with or separate from the feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

Many cell cultures incorporate a carbon source, like glucose, as a substrate feed in the cell culture during fermentation. Thus, in one embodiment, the method of cultivating the microorganism comprises a feed comprising a carbon source. The carbon source containing feed can comprise a defined or a complex carbon source as described in detail herein, or a mixture thereof.

The fermentation time, pH, conductivity, temperature, or other specific fermentation conditions may be applied according to standard conditions known in the art. In one embodiment, the fermentation conditions are adjusted to obtain maximum yields of the protein of interest.

In one embodiment, the temperature of the fermentation broth during fermentation is 30° C. to 45° C.

In one embodiment, the pH of the fermentation medium is adjusted to pH 6.5 to 9.

In one embodiment, the conductivity of the fermentation medium is after pH adjustment 0.1 100 mS/cm.

In one embodiment, the fermentation time is for 1-200 hours.

In one embodiment, fermentation is carried out with stirring and/or shaking the fermentation medium. In one embodiment, fermentation is carried out with stirring the fermentation medium with 50-2000 rpm.

In one embodiment, oxygen is added to the fermentation medium during cultivation, including, but not limited to, by stirring and/or agitation or by gassing, including but not limited to gassing with 0 to 3 bar air or oxygen. In one embodiment, fermentation is performed under saturation with oxygen.

In one embodiment, the fermentation medium and the method using the fermentation medium is for fermentation in industrial scale. In one embodiment, the fermentation medium of the present description may be useful for any fermentation having culture media of at least 20 litres, at least 50 litres, at least 300 litres, or at least 1000 litres.

In one embodiment, the fermentation method is for production of a protein of interest at relatively high yields, including, but not limited to, the protein of interest being expressed in an amount of at least 2 g protein (dry matter)/kg untreated fermentation medium, at least 3 g protein (dry matter)/kg untreated fermentation medium, of at least 5 g protein (dry matter)/kg untreated fermentation medium, at least 10 g protein (dry matter)/kg untreated fermentation medium, or at least 20 g protein (dry matter)/kg untreated fermentation medium.

Tolerance is to be understood as the ability of an organism to perform its normal functions at a substantial level, for example growth of the organism at a normal or somewhat reduced speed. Toxic substance like terpenes may result in substantially reduced growth or stop of growth or even kill the organism, depending on their toxicity and dosage. Improved tolerance to a toxic substance such as terpene will allow an organisms to perfom better at a dosage that normally has more sever effects on the organism.

In a preferred embodiment the homolog of a protein X is the one or more protein(s) corresponding in function and/or sequence to protein X in another organism than the organism protein X is originally found.

Activity of a protein of interest is to be understood as the normal biological function of said protein. Inactivation is to be understood in that said activity is not present to at the same normal level, but substantially lower or entirely absent. The abundance of said protein of interest at normal levels is required for the normal biological function as well. If the abundance of said protein of interest is reduced substantially, the biological function and hence overall activity will be reduced. If the protein(s) of interest are absent, e.g. since the gene encoding it has been made non-functional, has been deleted in part or full, has been knocked-out or its expression is prevented, the biological function is sooner or later abolished or no longer present in the organism.

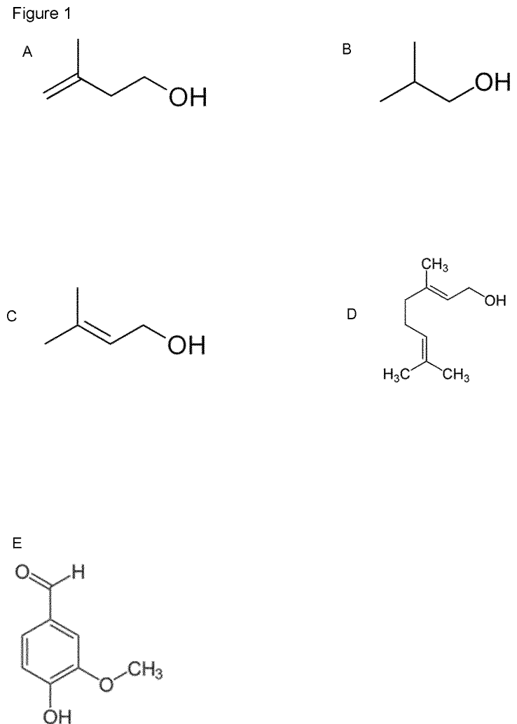

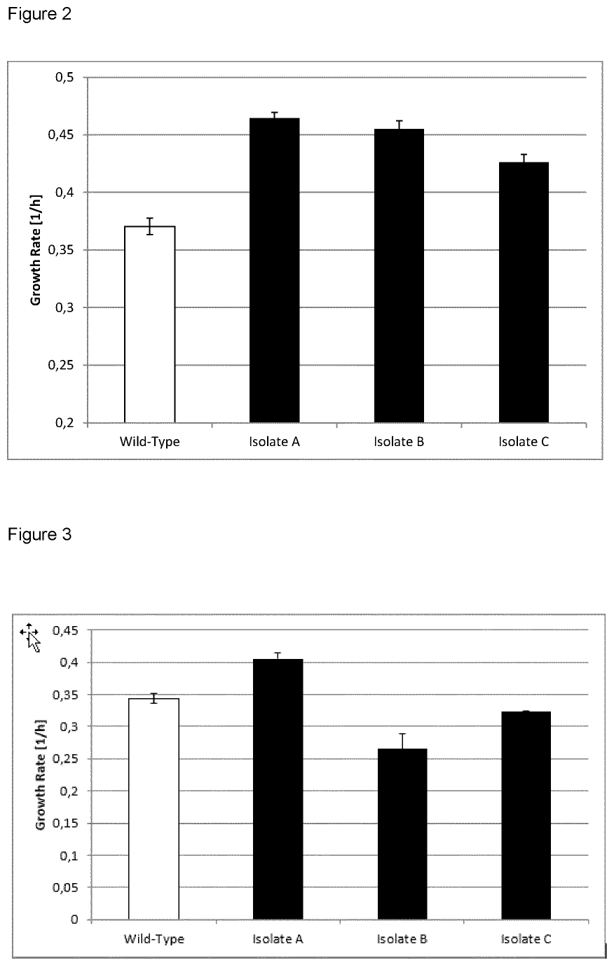

In a preferred embodiment terpene compounds are preferably C4 and C5 alcohols, substances with a log P value of 2.0 or less, preferably 1.5 or less and/or solubility in water of at least 1.0 g/l, preferably 1.5 g/I or more, shown in FIG. 1 and/or any of these compounds: isoprenol, prenol, butanol, isobutanol, Vanillin.

In another embodiment terpene compounds includes Geraniol, Citral, (−)-Carvone, Linalool, Farnesol, Limonene and Menthol.

In a preferred embodiment the organism with increased tolerance to terpenes and/or useful in the methods of the invention comprises a protein that shares the first 47 amino acids with the protein of SEQ ID NO: 2 or a homolog of SEQ ID NO: 2 in that organism, but either does not share any substantial identity from the amino acid that corresponds to position 48 of SEQ ID NO: 2 onwards; or it is shortened compared to the unmodified homolog of SEQ ID NO: 2 or SEQ NO:2. in the part following the amino acid corresponding to positions 1 to 47 of SEQ ID NO: 2.

1.-15. (canceled)

16. A modified organism with improved tolerance to one or more terpene compounds, wherein the modified organism has one or more alterations compared to a wildtype of the modified organism selected from the following group consisting of:
i. Increased levels or increased activity compared to the non-modified organism of protein of SEQ ID NO: 1 or a homolog thereof in the presence of one or more terpene compounds, preferably wherein the endogenous gene for the homolog of SEQ ID NO: 1 has been deleted and with recombinant expression of a gene encoding SEQ ID NO: 1 or a variant thereof, even more preferably wherein the recombinant expression of a gene encoding SEQ ID NO: 1 or a variant thereof is under a low to medium strength promoter or other control element;
ii. Absence, inactivation or reduced abundance of the protein of SEQ ID NO: 3 or a homolog thereof in the presence of one or more terpene compounds
iii. Presence of a mutated protein of the protein of SEQ ID NO: 4 or a homolog thereof in the presence of one or more terpene compounds, wherein the mutated protein of the protein of SEQ ID NO: 4 or a homolog thereof has a mutation at the position corresponding to the position 74 of SEQ ID NO: 4;
iv. In the presence of one or more terpene compounds presence of a mutated protein of the protein of SEQ ID NO: 5 or a homolog thereof preferably wherein the mutated protein of the protein of SEQ ID NO: 5 or a homolog thereof has a) a mutation at the position corresponding to the position 291 of SEQ ID NO: 5, and/or b) a mutation at the position corresponding to the position 274 of SEQ ID NO: 5 or thereafter wherein the mutated protein is shorter than the protein of SEQ ID NO:5 or the homolog thereof, or absence, inactivation or reduced abundance of the protein of SEQ ID NO: 5;
v. Absence, inactivation or reduced abundance of the protein of SEQ ID NO: 2 or a homolog thereof and absence, inactivation or reduced abundance of the protein of SEQ ID NO: 3 or a homolog thereof and presence of a mutated protein of the protein of SEQ ID NO: 2 or a homolog thereof in the presence of one or more terpene compounds, wherein the mutated protein of the protein of SEQ ID NO: 2 or a homolog thereof shares only the first 47 amino acids with the protein of SEQ ID NO: 2 or homolog thereof of the non-modified organism;
vi. Absence, inactivation or reduced abundance of the protein of SEQ ID NO: 2 or a homolog thereof in the presence of one or more terpene compounds
vii. Absence of the protein of SEQ ID NO: 2 or a homolog thereof and presence of a mutated protein of the protein of SEQ ID NO: 2 or a homolog thereof in the presence of one or more terpene compounds, wherein the mutated protein of the protein of SEQ ID NO: 2 or a homolog thereof has a mutation at the position corresponding to the position 48 of SEQ ID NO: 2;
viii. Presence of a mutated protein of the protein of SEQ ID NO: 2 or a homolog thereof in the presence of one or more terpene compounds, wherein the mutated protein of the protein of SEQ ID NO: 2 or a homolog thereof has a mutation at the position corresponding to the position 48 of SEQ ID NO: 2;
ix. Presence of a mutated protein of the protein of SEQ ID NO: 6 or a homolog thereof in the presence of one or more terpene compounds, wherein the mutated protein of the protein of SEQ ID NO: 6 or a homolog thereof has a mutation at the position corresponding to the position 96 of SEQ ID NO: 6, preferably the mutation is a mutation replacing a Valine with Glutamic acid, and/or a mutation at the position corresponding to the position 67 of SEQ ID NO: 6, preferably replacing a Glycine with a Serine;
x. Absence, inactivation or reduced abundance of the protein of SEQ ID NO: 6 or a homolog thereof in the presence of one or more terpene compounds;
xi. absence, inactivation or reduced abundance of the protein of SEQ ID NO: 8 or a homolog thereof, in the presence of one or more terpene compounds;
xii. absence, inactivation or reduced abundance of the protein of SEQ ID NO 9 or a homolog thereof in the presence of one or more terpene compounds;
xiii. absence, inactivation, increased activity or reduced abundance of the protein of SEQ ID NO 7 or a homolog thereof in the presence of one or more terpene compounds;
xiv. any combination of the previous i to xiii;
wherein the tolerance is improved compared to a non-modified organism.

17. Method for increasing the tolerance to one or more terpene compounds of a modified organism compared to a non-modified organism, including the steps of creating the modified organism according to claim 16 and optionally maintaining said modified organism.

18. Method for production of one or more terpene compounds using an organism, including the steps of creating the modified organism according to claim 16, maintaining said modified organism in the presence of one or more terpene compounds under conditions suitable for the modified organism to grow and produce said one or more terpene compound and optionally separating the one or more terpene compounds from said modified organism.

19. Method of claim 16, wherein the modified organism comprises a) a knock-out or a deletion in part or full of the gene encoding for the protein the protein of SEQ ID NO: 3 or a homolog thereof, a knock-out or b) a deletion in part or full of the gene encoding for the protein of SEQ ID NO: 2 or a homolog thereof, or c) presence of a mutated protein of the protein of SEQ ID NO: 2 or a homolog thereof in the presence of one or more terpene compounds, wherein the mutated protein of the protein of SEQ ID NO: 2 or a homolog thereof shares only the first 47 amino acids with the protein of SEQ ID NO: 2 or homolog thereof of the non-modified organism, or any combination of a) to c).

20. The method of claim 16 including the step of downregulating the expression of the gene encoding the protein of SEQ ID NO: 6 or a homolog thereof, deleting the gene encoding the protein of SEQ ID NO: 6 or a homolog thereof or knock out the gene encoding the protein of SEQ ID NO: 6 or a homolog thereof

21. A method for the production of monoterpene esters including production of one or more monoterpenes according to the methods of claim 18, and esterifying at least one monoterpene to a monoterpene ester, and optionally separation of the one or more monoterpene ester.

22. Method for increasing tolerance to Vanillin of a modified organism compared to a nonmodified organism including the step of in a modified organism expressing of or generating a DNA sequence encoding a protein that shares only the first 47 amino acids with the protein of SEQ ID NO: 2, wherein the modified organism has the further characteristic that the proteins of SEQ ID NOs: 1 and/or 2 or homologs thereof are absent, inactive or substantially reduced.

23. Use of a deregulated protein of SEQ ID NO: 2 or a homolog thereof to increase growth of modified organisms in the presence of one or more terpene compounds, preferably one or more terpenes or one or more terpene esters.

24. The modified organism of claim 16 wherein the mutated or deregulated protein of SEQ ID NO: 2 or homolog thereof has a mutation of the histidine residue corresponding to the position 48 of SEQ ID NO: 2 resulting in a frameshift, preferably a frameshift shortening the resulting protein compared to the protein of SEQ ID NO: 2.

25. The modified organism of claim 16, wherein any of the sequences of SEQ ID NOs: 1 to 6 are mutated to carry the mutations as shown in table 3 for the respective protein.

26. The methods, the use, the mutated protein or the modified organism of claim 16 wherein the tolerance to isoprenol, prenol, butanol, isobutanol, Vanillin, Geraniol, santalene, valencene, sclareol, artemisinic alcohol, artemisinic acid and/or Citral is increased compared to a nonmodified organism.

27. The methods, the use, the mutated protein or the modified organism of claim 16 wherein the tolerance to isoprenol, prenol, butanol, isobutanol and/or Vanillin is increased compared to a non-modified organism.

28. The methods, the use or the modified organism of claim 16 wherein at least one terpene compound has a log P value of 2.0 or less, preferably 1.5 or less

29. The methods, the use or the modified organism of claim 16 wherein the solubility in water of at least one terpene compound is at least 1.0 g/l, preferably 1.5 g/l or more.

30. The methods, the use or the modified organisms of claim 16 wherein at least one terpene compound is a monoterpene alcohol or a C4 and C5 alcohol.
